(19) United States
(12) Patent Application Publication
(10) Pub. No.: US 2006/0217375 A1
(43) Pub. Date: Sep. 28, 2006

(54) New compounds

(76) Inventors: Emma Barker, Fordham (GB); Annika Jenmalm Jensen, Uppsala (SE); Erik Nordling, Solna (SE); Andrew Proud, Kent (GB); Martin Slater, Kent (GB); Michael Weber, Uppsala (SE); Lars Tedenborg, Uppsala (SE)

Correspondence Address: FISH & RICHARDSON PC, P.O. BOX 1022, MINNEAPOLIS, MN, 55440-1022, US

(21) Appl. No.: 11/318,126

(22) Filed: Dec. 22, 2005

Related U.S. Application Data
(60) Provisional application No. 60/653,803, filed on Feb. 17, 2005.

(30) Foreign Application Priority Data
Dec. 23, 2004 (SE) 0403160-5

(51) Int. Cl. A61K 31/5377; A61K 31/496; C07D 491/10

(52) U.S. Cl. 514/232.5; 514/254.11; 544/70; 544/230

(57) ABSTRACT

The present invention relates to compounds of Formula (I): 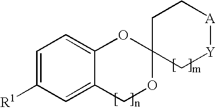
wherein R¹, A, Y, n and m are as described herein, processes for preparing the compounds, pharmaceutical compositions comprising the compounds, and use of the compounds and compositions in the prophylaxis or treatment of orexin-1 receptor-related disorders and orexin-2 receptor-related disorders. Examples of such disorders are obesity and related disorders such as diabetes type II, dyslipidemia and the metabolic syndrome, cardiovascular diseases such as atherosclerotic vascular disease, angina pectoris, myocardial infarction and stroke, drug addiction, and sleeping disorders.

### RELATED APPLICATION INFORMATION

This application claims priority to U.S. provisional application Ser. No. 60/653,803, filed Feb. 17, 2005, and to Swedish application serial no. 0403160-5, filed Dec. 23, 2004.

### TECHNICAL FIELD

The present invention relates to novel compounds, to pharmaceutical compositions comprising the compounds, to processes for their preparation, the use of the compounds for the preparation of medicaments against orexin-1 receptor-related disorders and orexin-2 receptor-related disorders, and methods for the prophylaxis and treatment of orexin-1 receptor-related disorders and orexin-2 receptor-related disorders.

### BACKGROUND ART

The orexins/hypocretins are two neuropeptides encoded by the common precursor preproorexin. Mammalian orexin-A is a 33 amino acid peptide with two intrachain disulfide bonds, whereas orexin-B is a 28 amino acid linear peptide (Sakurai et al. (1998) Cell 92: 573-585). The orexins are mainly expressed in the lateral hypothalamus, an area known to be active in the regulation of food intake, but since orexin neurons project widely to different brain areas, other physiological roles are also implicated.

Two orexin receptor subtypes belonging to the G protein-coupled receptor class (GPCR) have been identified in mammals, and they have 64% amino acid identity with each other (Sakurai et al. (1998) Cell 92: 573-585). The Orexin-1 receptor (OX-1R; HCRTR¹) has one order of magnitude greater affinity for orexin-A compared with orexin-B. In contrast, both orexin-A and orexin-B binds to the Orexin-2 receptor (OX-2R; HCRTR²) with equal affinity.

Studies with in situ hybridization have shown that OX-1R and OX-2R have different and complementary distribution (Trivedi et al. (1998) FEBS Lett. 438: 71-75); Marcus et al. (2001) J. Comp. Neurol. 435: 6-25). It has been shown that alpha- and beta-cells in pancreatic islets express orexin-A, and that both cell types express OX-1R receptors (Ouedraogo et al. (2003) Diabetes 52: 111-117). Orexin-A increases glucagon secretion and decreases glucose-stimulated insulin release from isolated islets. Furthermore, orexin-A infusion increases plasma glucagon and glucose levels and decreases plasma insulin in fasted rats.

The lateral hypothalamic area (LHA) has long been known to affect hunger and ingestive behavior as well as the regulation of sleep-wakefulness (Bernardis & Bellinger (1996) Neurosci. Biobehav. Rev. 20: 189-287). Administration of orexin into the LHA of rodents in the early light-phase stimulates food intake in a dose-dependent manner (Sakurai et al. (1998) Cell 92: 573-585; Haynes et al. (1999) Peptides 20: 1099-1105; Yamanaka et al. (2000) Brain Res. 859: 404-409.). A study has reported that prolonged continuous administration (i.c.v.) of orexin-A increases daytime food intake, and reduces sleep, but does not have any effect on body weight gain (Yamanaka et al. (1999) Brain Res. 849: 248-252). The effects of orexin-A seem to be dependent on the circadian rhythm. Orexin-A has also been shown to increase energy expenditure in a dose-dependent manner when administered i.c.v. to anesthetized rats (Wang et al. (2001) Neurosci. Lett. 315: 49-52). Orexin-A stimulated feeding can be partially blocked by NPY-Y1 receptor antagonists (Yamanaka et al. (2000) Brain Res. 859: 404-409), and leptin administration can only partially block orexin-induced food intake in rats (Zhu et al. (2002) Physiol. Behav. 77: 251-257). Orexin neurons have been shown to project to the arcuate nucleus and to innervate NPY neurons (Broberger et al. (1998) J. Comp. Neurol. 402: 460-474). I.c.v. injection of orexin elicits c-Fos expression in these neurons suggesting that orexin-induced feeding may occur in part via NPY pathways (Yamanaka et al. (2000) Brain Res. 859: 404-409).

The orexin system is strongly implicated in narcolepsy, a disease characterized by disorganization of the vigilance state. Patients suffer from excessive daytime sleepiness, cataplexy, and disturbed REM sleep patterns. Recently, it was shown that the activity of isolated orexin neurons is inhibited by glucose and leptin and stimulated by ghrelin, and the orexin expression in normal as well as ob/ob mice correlates negatively with changes in blood glucose, leptin, and food intake. Orexin neuron ablated mice fail to respond to fasting with increased vigilance and activity. This indicates a link between energy balance and orexin-mediated arousal (Yamanaka et al. (2003) Neuron 38: 701-713). Mice with a genetic ablation of the OX-2R exhibit several of the characteristics of narcolepsy (Tokita et al. (2001) Sleep 24: A20-21; Willie et al. (2003) Neuron 38: 715-730). In contrast, OX-1R ablated mice do not have an overt behavioral phenotype and exhibit only an increased fragmentation of sleep-wake cycles (Kisanuky et al. (2001) Sleep 24: A22).

The physiological relevance of the OX-1R system on feeding is supported by data that have emerged from the use of specific and potent OX-1R antagonists (Haynes et al. (2000) Regul Pept. 96: 45-51; Duxon et al. (2001) Psychopharmacology (Berl). 153: 203-209; Smart et al. (2001) Br. J. Pharmacol. 132: 1179-1182; Smart et al. (2002) Eur. J. Pharmacol. 440: 199-212; Langmead et al. (2004) Br. J. Pharmacol. 141: 340-346). The specific OX-1R antagonist, SB-334867, developed by GlaxoSmithKIine, when given to rats i.p. advances the behavioral satiety sequence increasing the time the animals spend resting (Rodgers et al. (2001) Eur. J. Neurosci. 13: 1444-1452.). The anti-obesity effect of SB-334867 has also been examined in ob/ob mice (Haynes et al. (2002) Regul. Pept. 104:153-159). When these mice were treated with SB-334867 (30 mg/kg i.p. once daily for 7 days plus twice daily for another 7 days) cumulative food intake and body weight gain was suppressed over this period. Total fat mass was reduced, whereas lean body mass was unchanged.

Georgescu et al. ((2003 J. Neurosci. 23:3106) reported that orexin knock-out mice develop attenuated morphine dependence, as indicated by reduced withdrawal symptoms when treated with a morphine antagonist. Georgescu et al. state that this result suggests that regulation of orexin neurons may be an important contributor to morphine physical dependence and withdrawal.

WO 2004/111560, published 23 Dec. 2004 describes compound libraries said to be useful for identifying compounds that bind to G-protein coupled receptors.

### DISCLOSURE OF THE INVENTION

It has surprisingly been found that spiro[benzodioxane] compounds of the general Formula I are active as antagonists of the orexin-1 receptor, in particular the human orexin-1 receptor, and potentially useful in the prophylaxis and treatment of orexin-1 receptor-related disorders and orexin-2 receptor-related disorders.

In a first aspect this invention provides a compound of the Formula I, wherein 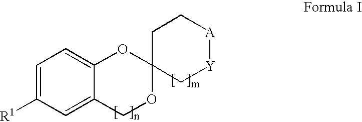
n and m are, independently, 0 or, preferably, 1;

A and Y are independently CH₂, O or NR², wherein R² is H or C₁-C₆ alkyl, provided that one of A and Y is CH₂, and the other one is O or NR², and provided that when m is 0, then Y is CH₂;

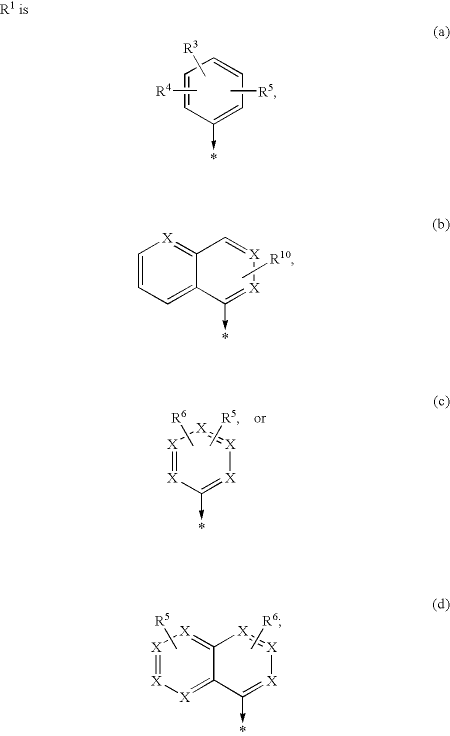

X is CH or N,

provided that when R¹ is (c) or (d), not more than two of the groups X are N;

R³ and R⁴ are independently C₁-C₆ alkoxy;

R⁵ is H, halogen, C₁-C₆ alkyl, or C₁-C₆ alkoxy;

R⁶, which is bonded to R¹ in a position wherein X is CH, is

(a) —R⁷—CO—(CH₂)ₚ—Ar,

(b) —R⁷—CO—CH₂—O—Ar,

(c) —R⁷—CO—CH₂—S—Ar,

(d) —R⁷—(CH₂)ₚ—CO—NH—R⁸,

(e) —R⁷—(CH₂)ₚ—CO—R⁹, (f) —R⁷—(CH₂)ₚ—R⁹, or

(g) —C(O)—NH—(CH₂)ₚ—Ar,

wherein p is an integer 1 or 2;

R⁷ is O or NH;

R⁸ is H or Ar;

Ar is aryl or heteroaryl,

Ar being optionally independently substituted with one or more of halogen, C₁-C₆ alkyl, C₁-C₆ alkoxy, di-(C₁-C₃)alkylamino, or when Ar is indole, MeSO₂;

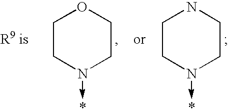

R¹⁰, which is bonded to R¹ in a position wherein X is CH, is H or NH₂;

and pharmaceutically acceptable salts, hydrates, solvates, geometrical isomers, tautomers, optical isomers, N-oxides and prodrug forms thereof.

Preferably, A is NH and Y is CH₂.

Preferably, R¹ is (c) or (d), such as phenyl or quinoline, in particular phenyl, and substituted with R⁶.

When R⁶ is (a), (b) or (c), then R⁷ is preferably NH. When R⁶ is (d), (e) or (f), then R⁷ is preferably O. When R⁶ is (a), (d), (e) or (f), then p is preferably 1. When R⁶ is (d), then R⁸ is preferably Ar.

Ar is preferably phenyl or indole. Further, Ar is preferably independently substituted with one or more C₁-C₆ alkoxy groups.

Examples of preferred compounds of Formula I include those wherein R⁶ is any one of the following groups:

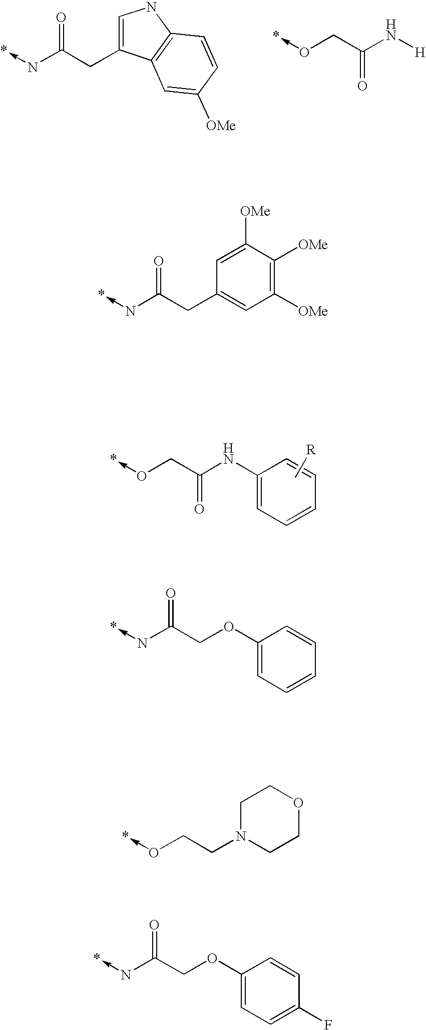

wherein there can be 1, 2, or 3 R that are independently selected from: H, halogen, C₁-C₆ alkyl, or C₁-C₆ alkoxy or haloalkyl.

Preferred compounds of Formula I are those having the Formula II: 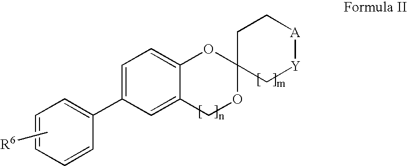
wherein A, Y, R⁶, n, and m are as defined above. Thus, in Formula II, n and m are, independently, 0 or, preferably, 1;

A and Y are independently CH₂, O or NR², wherein R² is H or C₁-C₆ alkyl, provided that one of A and Y is CH₂, and the other one is O or NR², and provided that when m is 0, then Y is CH₂;

R⁶ is selected from:

(a) —R⁷—CO—(CH₂)ₚ—Ar,

(b) —R⁷—CO—CH₂—O—Ar,

(c) —R⁷—CO—CH₂—S—Ar,

(d)—R⁷—(CH₂)ₚ—CO—NH—R⁸,

(e) —R⁷—(CH₂)ₚ—CO—R⁹, and

(f) —R⁷—(CH₂)ₚ—R⁹.

More preferred compounds are those having the Formula III: 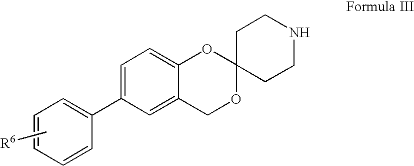
wherein R⁶ is selected from:

(a) —R⁷—CO—(CH₂)ₚ—Ar,

(b) —R⁷—CO—CH₂—O—Ar,

(c) —R⁷—CO—CH₂—S—Ar,

(d) —R⁷—(CH₂)ₚ—CO—NH—R⁸,

(e) —R⁷—(CH₂)ₚ—CO—R⁹, and

(f) —R⁷—(CH₂)ₚ—R⁹,

Examples of preferred compounds of formula I are the following:
5-(3,4,5-trimethoxyphenyl)spiro[1,3-benzodioxole-2,4′-piperidine];
5-(2′,3′,5′,6′-tetrahydro-4H-spiro[1,3-benzodioxine-2,4′-pyran]-6-yl)quinoline;
6-(3,4,5-trimethoxyphenyl)-4H-spiro[1,3-benzodioxine-2,3′-piperidine];
N-(2,6-dimethylphenyl)-2-[3-(4H-spiro[1,3-benzodioxine-2,4′-piperidin]-6-yl)phenoxy]acetamide;
N-phenyl-2-[3-(4H-spiro[1,3-benzodioxine-2,4′-piperidin]-6-yl)phenoxy]acetamide;
2-(5-methoxy-1H-indol-3-yl)-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-2-(3,4,5-trimethoxyphenyl)acetamide;
2-(4-fluorophenoxy)-N-[3-(4H-spiro[1,3-benzodioxine-2,4′-piperidin]-6-yl)phenyl]acetamide;
1-(4H-spiro[1,3-benzodioxine-2,4′-piperidin]-6-yl)isoquinolin-3-amine;
2-[2-(4H-spiro[1,3-benzodioxine-2,4′-piperidin]-6-yl)phenoxy]acetamide;
2-[3-(4H-spiro[1,3-benzodioxine-2,4′-piperidin]-6-yl)phenoxy]-N-[2-(trifluoromethyl)phenyl]acetamide;
6-[3-(2-morpholin-4-ylethoxy)phenyl]-4H-spiro[1,3-benzodioxine-2,4′-piperidine];
6-(2,4-dimethoxyphenyl)-4H-spiro[1,3-benzodioxine-2,4′-piperidine];
6-(3,4,5-trimethoxyphenyl)-4H-spiro[1,3-benzodioxine-2,4′-piperidine];
6-quinolin-5-yl-4H-spiro[1,3-benzodioxine-2,4′-piperidine];
N-[3-(4H-spiro[1,3-benzodioxine-2,4′-piperidin]-6-yl)phenyl]pyrazine-2-carboxamide;
2-(3,4-dimethoxyphenyl)-N-[3-(4H-spiro[1,3-benzodioxine-2,4′-piperidin]-6-yl)phenyl]acetamide;
N-[2-(3,4-dimethoxyphenyl)ethyl]-3-(4H-spiro[1,3-benzodioxine-2,4′-piperidin]-6-yl)benzamide;
3-(4H-spiro[1,3-benzodioxine-2,4′-piperidin]-6-yl)-N-(3,4,5-trimethoxybenzyl)benzamide
2-(2,3-dimethoxyphenyl)-N-[3-(4H-spiro[1,3-benzodioxine-2,4′-piperidin]-6-yl)phenyl]acetamide;
2-(2,4-dimethoxyphenyl)-N-[3-(4H-spiro[1,3-benzodioxine-2,4′-piperidin]-6-yl)phenyl]acetamide;
3-(1H-indol-3-yl)-N-[3-(4H-spiro[1,3-benzodioxine-2,4′-piperidin]-6-yl)phenyl]propanamide;
2-(5-methoxy-2-methyl-1H-indol-3-yl)-N-[3-(4H-spiro[1,3-benzodioxine-2,4′-piperidin]-6-yl)phenyl]acetamide;
2-(2-methyl-1H-indol-3-yl)-N-[3-(4H-spiro[1,3-benzodioxine-2,4′-piperidin]-6-yl)phenyl]acetamide;
2-(7-methyl-1H-indol-3-yl)-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-3-(3,4,5-trimethoxyphenyl)propanamide;
3-phenoxy-N-[3-(4H-spiro[1,3-benzodioxine-2,4′-piperidin]-6-yl)phenyl]propanamide
2-(3,5-dimethoxyphenyl)-N-[3-(4H-spiro[1,3-benzodioxine-2,4′-piperidin]-6-yl)phenyl]acetamide;
2-[4-(dimethylamino)phenyl]-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-2-(2,3,4-trimethoxyphenyl)acetamide;
2-(4-hydroxy-3,5-dimethoxyphenyl)-N-[3-(4H-spiro[1,3-benzodioxine-2,4′-piperidin]-6-yl)phenyl]acetamide;
2-(4-hydroxy-3-methoxyphenyl)-N-[3-(4H-spiro[1,3-benzodioxine-2,4′-piperidin]-6-yl)phenyl]acetamide
N-[3-(4H-spiro[1,3-benzodioxine-2,4′-piperidin]-6-yl)phenyl]-2-[3-(trifluoromethyl)phenyl]acetamide;
2-(6-methoxy-1-benzofuran-3-yl)-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-N′-(3,4,5-trimethoxyphenyl)urea;
N-[3-(4H-spiro[1,3-benzodioxine-2,4′-piperidin]-6-yl)phenyl]-N′-(3,4,5-trimethoxybenzyl)urea;
N-(2-methylquinolin-6-yl)-N′-[3-(4H-spiro[1,3-benzodioxine-2,4′-piperidin]-6-yl)phenyl]urea;
N-[2-(5-methoxy-1H-indol-3-yl)ethyl]-3-(4H-spiro[1,3-benzodioxine-2,4′-piperidin]-6-yl)benzamide;
2-(5-methoxy-1H-indol-3-yl)-N-[2-methoxy-5-(4H-spiro[1,3-benzodioxine-2,4′-piperidin]-6-yl)phenyl]acetamide;
N-[2-methoxy-5-(4H-spiro[1,3-benzodioxine-2,4′-piperidin]-6-yl)phenyl]-2-(3,4,5-trimethoxyphenyl)acetamide;
3-(3,4-dimethoxyphenyl)-N-[2-methoxy-5-(4H-spiro[1,3-benzodioxine-2,4′-piperidin]-6-yl)phenyl]propanamide;
2-(5-hydroxy-1H-indol-3-yl)-N-[3-(4H-spiro[1,3-benzodioxine-2,4′-piperidin]-6-yl)phenyl]acetamide;
N-[2-(1H-indol-3-yl)ethyl]-3-(4H-spiro[1,3-benzodioxine-2,4′-piperidin]-6-yl)benzamide
N-[2-(6-methoxy-1H-indol-3-yl)ethyl]-3-(4H-spiro[1,3-benzodioxine-2,4′-piperidin]-6-yl)benzamide;
N-[2-(5-chloro-1H-indol-3-yl)ethyl]-3-(4H-spiro[1,3-benzodioxine-2,4′-piperidin]-6-yl)benzamide;
N-[2-(5-fluoro-1H-indol-3-yl)ethyl]-3-(4H-spiro[1,3-benzodioxine-2,4′-piperidin]-6-yl)benzamide;
N-[2-(5-methoxy-1H-indol-3-yl)ethyl]-5-(4H-spiro[1,3-benzodioxine-2,4′-piperidin]-6-yl)nicotinamide;
N-[5-(4H-spiro[1,3-benzodioxine-2,4′-piperidin]-6-yl)pyridin-3-yl]-2-(3,4,5-trimethoxyphenyl)acetamide;
2-(5-methoxy-1H-indol-3-yl)-N-[5-(4H-spiro[1,3-benzodioxine-2,4′-piperidin]-6-yl)pyridin-3-yl]acetamide;
N-[2-(5-methoxy-1H-indol-3-yl)ethyl]-4-(4H-spiro[1,3-benzodioxine-2,4′-piperidin]-6-yl)pyridine-2-carboxamide;
2-fluoro-5-(4H-spiro[1,3-benzodioxine-2,4′-piperidin]-6-yl)-N-(3,4,5-trimethoxybenzyl)benzamide;
2-fluoro-N-[2-(5-methoxy-1H-indol-3-yl)ethyl]-5-(4H-spiro[1,3-benzodioxine-2,4′-piperidin]-6-yl)benzamide;
2-(1H-benzimidazol-1-yl)-N-[3-(4H-spiro[1,3-benzodioxine-2,4′-piperidin]-6-yl)phenyl]acetamide;
2-(5-methyl-2-phenyl-1,3-thiazol-4-yl)-N-[3-(4H-spiro[1,3-benzodioxine-2,4′-piperidin]-6-yl)phenyl]acetamide;
2-(4-tert-butylphenyl)-N-[3-(4H-spiro[1,3-benzodioxine-2,4′-piperidin]-6-yl)phenyl]acetamide;
2-(5-ethyl-1H-indol-3-yl)-N-[3-(4H-spiro[1,3-benzodioxine-2,4′-piperidin]-6-yl)phenyl]acetamide;
3-(1H-benzimidazol-1-yl)-N-[3-(4H-spiro[1,3-benzodioxine-2,4′-piperidin]-6-yl)phenyl]propanamide;
2-(2H-1,2,3-benzotriazol-2-yl)-N-[3-(4H-spiro[1,3-benzodioxine-2,4′-piperidin]-6-yl)phenyl]acetamide;
3-(2-phenyl-1H-imidazol-1-yl)-N-[3-(4H-spiro[1,3-benzodioxine-2,4′-piperidin]-6-yl)phenyl]propanamide;
3-(2,3-dihydro-1-benzofuran-5-yl)-N-[3-(4H-spiro[1,3-benzodioxine-2,4′-piperidin]-6-yl)phenyl]propanamide;
2-(3-pyridin-3-ylphenyl)-N-[3-(4H-spiro[1,3-benzodioxine-2,4′-piperidin]-6-yl)phenyl]acetamide;
N-[3-(1′-isopropyl-4H-spiro[1,3-benzodioxine-2,4′-piperidin]-6-yl)phenyl]-2-(3,4,5-trimethoxyphenyl)acetamide;
3-(3-chloro-4-methoxyphenyl)-N-[3-(4H-spiro[1,3-benzodioxine-2,4′-piperidin]-6-yl)phenyl]propanamide;
2-(1H-benzimidazol-2-yl)-N-[3-(4H-spiro[1,3-benzodioxine-2,4′-piperidin]-6-yl)phenyl]acetamide;
3-(1H-1,2,3-benzotriazol-1-yl)-N-[3-(4H-spiro[1,3-benzodioxine-2,4′-piperidin]-6-yl)phenyl]propanamide;
3-(1H-indazol-1-yl)-N-[3-(4H-spiro[1,3-benzodioxine-2,4′-piperidin]-6-yl)phenyl]propanamide;
2-(2-methyl-1H-benzimidazol-1-yl)-N-[3-(4H-spiro[1,3-benzodioxine-2,4′-piperidin]-6-yl)phenyl]acetamide;
3-(2-methyl-1H-benzimidazol-1-yl)-N-[3-(4H-spiro[1,3-benzodioxine-2,4′-piperidin]-6-yl)phenyl]propanamide;
2-pyridin-2-yl-N-[3-(4H-spiro[1,3-benzodioxine-2,4′-piperidin]-6-yl)phenyl]acetamide;
2-pyridin-4-yl-N-[3-(4H-spiro[1,3-benzodioxine-2,4′-piperidin]-6-yl)phenyl]acetamide;
3-(1H-benzimidazol-2-yl)-N-[3-(4H-spiro[1,3-benzodioxine-2,4′-piperidin]-6-yl)phenyl]propanamide;
N-methyl-N-(2-oxo-2-{[3-(4H-spiro[1,3-benzodioxine-2,4′-piperidin]-6-yl)phenyl]amino}ethyl)benzamide;
N-[2-(3,4-dimethoxyphenyl)ethyl]-2-methyl-3-(4H-spiro[1,3-benzodioxine-2,4′-10 piperidin]-6-yl)benzamide; and
2-methyl-3-(4H-spiro[1,3-benzodioxine-2,4′-piperidin]-6-yl)-N-(3,4,5-trimethoxybenzyl)benzamide.

Included in the invention is a process for synthesis of the compounds of Formula I. The said compounds can be synthesized by methods known in the art, as generally described in the following experimental section.

Another object of the present invention is a compound as mentioned above for use in therapy, especially for use in the prophylaxis or treatment of an orexin-1 receptor-related disorder or an orexin-2 receptor-related disorder.

Another object of the present invention is a pharmaceutical formulation comprising a compound as mentioned above as active ingredient, in combination with a pharmaceutically acceptable diluent or carrier, especially for use in the prophylaxis or treatment of an orexin-1 receptor-related disorder or an orexin-2 receptor-related disorder.

Another object of the present invention is a method for treating a human or animal subject suffering from an orexin-1 receptor-related disorder. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the orexin-1 receptor-related disorder or an orexin-2 receptor-related disorder. Identifying a subject in need of such treatment can be in the judgment of a subject or a health care professional and can be subjective (e.g., opinion) or objective (e.g., measurable by a test or diagnostic method).

Another object of the present invention is a method for the prophylaxis of an orexin-1 receptor-related disorder or an orexin-2 receptor-related disorder, which comprises administering to a subject in need of such treatment an effective amount of a compound as mentioned above.

Another object of the present invention is a method for modulating (e g, promoting or inhibiting) orexin-1 receptor activity or orexin-2 receptor related activity, which comprises administering to a subject in need of such treatment an effective amount of a compound as mentioned above.

Another object of the present invention is a method for suppressing food intake, which comprises administering to a subject in need of such treatment an effective amount of a compound as mentioned above.

Another object of the present invention is a method for suppressing appetite, which comprises administering to a subject in need of such treatment an effective amount of a compound as mentioned above.

Another object of the present invention is a method for reducing weight, which comprises administering to a subject in need of such treatment an effective amount of a compound as mentioned above.

Another object of the present invention is a method for reducing weight gain, which comprises administering to a subject in need of such treatment an effective amount of a compound as mentioned above.

Another object of the present invention is an method for treating drug or alcohol addiction (e.g., addiction to an opiate such a morphine) or reducing the severity of one or more symptoms of drug withdrawal, which comprises administering to a subject in need of such treatment an effective amount of a compound as mentioned above.

Another object of the present invention is the use of a compound as mentioned above for the manufacture of a medicament for use in the prophylaxis or treatment of an orexin-1 receptor-related disorder or an orexin-2 receptor-related disorder.

Examples of orexin-1 receptor-related disorders and orexin-2 receptor-related disorders are obesity and related disorders such as diabetes type II, dyslipidemia and the metabolic syndrome; cardiovascular diseases such as atherosclerotic vascular disease, angina pectoris, myocardial infarction and stroke; and sleeping disorders such as insomnia or narcolepsy. The compounds according to the invention may also be useful for blocking the emetic response, i.e. useful in the treatment of nausea and vomiting.

The term “aryl” in the present description refers to a hydrocarbon ring system (monocyclic or bicyclic) having from 6 to 10 ring carbon atoms, and having at least one aromatic ring. Likewise, the term “aryloxy” refers to an aryl group bonded to an oxygen atom. Examples of aryls are phenyl, pentalenyl, indenyl, indanyl, isoindolinyl, chromanyl, naphthyl, fluorenyl, anthryl, phenanthryl and pyrenyl.

The term “heteroaryl” means in the present description a monocyclic, bi- or tricyclic aromatic ring system (only one ring need to be aromatic) having from 5 to 14, preferably 5 to 10 ring atoms such as 5, 6, 7, 8, 9 or 10 ring atoms (mono- or bicyclic), in which one or more of the ring atoms are other than carbon, such as nitrogen, sulfur, oxygen and selenium as part of the ring system. Likewise, the term heteroaryloxy refers to a heteroaryl group bonded to an oxygen atom.

Examples of heteroaryl rings are pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline (i e 2,3-dihydroindole), isoindoline (i.e. 1,3-dihydroisoindole), benzothiophene, benzofuran, isobenzofuran, benzoxazole, 2,1,3-benzoxadiazole, benzopyrazole; benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, pyrido[3,2-b]thiophene, tetralin, methylenedioxyindole, 2,3-dihydrobensofuran, 2,3-dihydrobensotiofen, 1,3-benzoxathiole, acridine, fenazine and xanthene.

Unless otherwise stated or indicated, the term “C₁₋₆-alkyl” (or alternatively “C₁-C₆ alkyl”) denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc.

Unless otherwise stated or indicated, the term “C₁₋₆ alkoxy” (or alternatively “C₁-C₆ alkoxy”) denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “halogen” shall mean fluorine, chlorine, bromine or iodine.

### EXPERIMENTAL METHODS

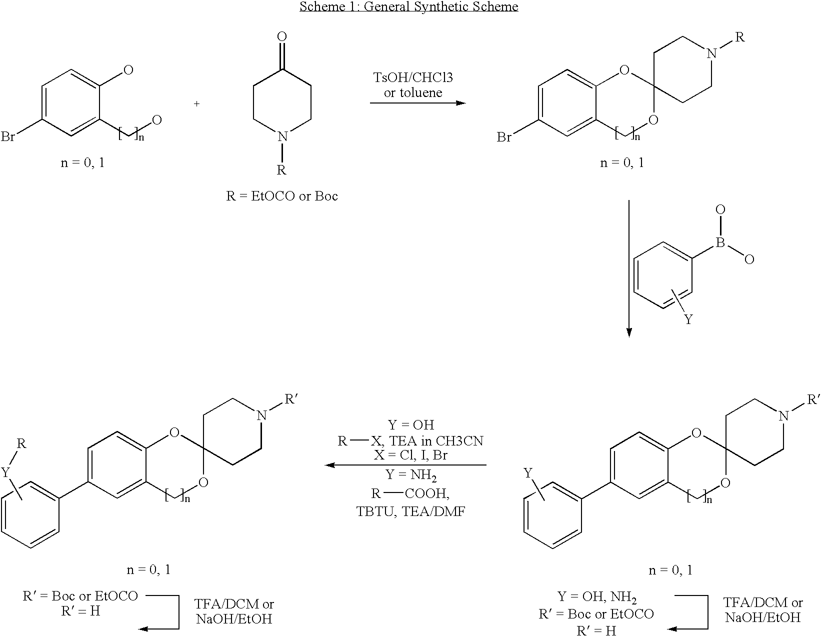

Synthesis commences with an acetalisation reaction of a hydroxyphenol derivative and a protected piperidinone to form the spiro[benzodioxane-piperidine] scaffold. Suzuki coupling with different boronic acids afforded the desired N-protected compounds, which were in some cases reacted further with electrophiles such as carboxylic acids and aliphatic halides.

All reagents were commercial grade and were used as received without further purification, unless otherwise specified. The chemicals were bought from Sigma-Aldrich (The old brickyard, New road, Gillingham, Dorset, SP8 4XT, UK), Lancaster (Eastgate, White Lund, Morecambe, Lancashire, LA3 3DY, UK), and Acros (Bishop Meadow road, Loughborough, leicestershire, LE11 5RG, UK). Commercially available anhydrous solvents were used for reactions conducted under inert atmosphere. Reagent grade solvents were used in all other cases, unless otherwise specified. Thin layer chromatography was carried out using pre-coated silica gel F-254 plates (thickness 0.25 mm). Column chromatography was performed on Matrex® silica gel 60 (35-70 micron) on Silica gel 60 (0.04-0.063 mm) (Merck) and on prepacked silica gel columns Redisep® (Isco). LC-MS was run on an LCD-MS (Agilent) with HP1000 HPLC. NMR was run on an Eclipse 270 (Jeol) instrument or on a Brucker Avance250 at 250 MHz. Chemical shifts are given in ppm using the solvent peak as standard. HPLC was run on HP1000 (Agilent) using System A: ACE 3 C8-column, 50×3 mm, 1 mL/min acetonitrile/water with 0.1% TFA at 40° C., System B: YMC-column or System C: Xterra column 3.5 μm C18, 3×50 mm with 0.01 M NH₄HCO₃, pH 10-CH₃CN gradient. HPLC was also run on using a Waters Xterra MS C18 column (100×4.6 mm, 5μ) eluting with a gradient of 5% CH₃CN in 95% water to 95% CH₃CN in 5% water (0.2% TFA buffer) over 3.5 min, then 95% CH₃CN in 5% water (0.2% TFA buffer) for a further 2.5 min at a flow rate of 3 ml/min on a Waters 600E or Gilson system with monitoring at 254 nm.

Preparative HPLC was performed on a Gilson system equipped with Xterra 5 μm, C8, 19×50 mm column using 0.05 M NH₄HCO₃, pH 10-CH₃CN gradient with a flow of 25 mL/min or ACE 5 C8 using acetonitrile/water with 0.1% TFA.

General Synthetic Procedure for Acetal Formation (A)

The diol (2 mmol), the ketone (2.6 mmol) in chloroform (15 mL) and TsOH monohydrate (38 mg, 0.2 mmol) was heated under reflux with a Dean-Stark condensor for 1-2 days. The solvent was evaporated and the residue was dissolved in EtOAc and washed with 2 M NaOH (2×5 mL) and brine. Flash chromatography on silica gel with ca 15% EtOAc/hexane gave the product.

General Synthetic Procedure for Suzuki Reaction (B1)

The arylbromide (0.25 mmol) and the boronic acid (0.30 mmol) were mixed with PPh₃ (10 mg, 0.0375 mmol) and NaHCO₃ (63 mg, 0.75 mmol) in 80% aq. EtOH (1.25 mL). Then Pd(OAc)₂ (ca 3 mg, 0.0125 mmol) was added and the mixture stirred at 90° C. for 17 h. 4 mL 80% EtOH was added, the mixture was filtered through Celite and most of the solvent was evaporated. The residue was added to DCM (15 mL) and 5% NaHCO₃ (5 mL). The organic phase was separated and washed with 5% NaHCO₃ and brine. The DCM-phase was dried and evaporated. Flash chromatography on 10-12 mL silica gel with 3-4% MeOH/DCM gave the product.

General Synthetic Procedure for Suzuki Reaction (B2)

The arylbromide and the boronic acid were mixed with PdCl₂dppf and NaHCO₃ in 80% aq. EtOH. The mixture stirred at 80° C. over night. The mixture was suspended in ethanol and filtered through Celite. The solvent was removed in vacuo. Purification was performed by preparative HPLC.

General Synthetic Procedure for Hydrolysis of Ethyl Carbamates (C)

The ethyl carbamate (36-80 mg) was dissolved in EtOH (1.3 mL) and 6 M NaOH (0.46 mL) was added. The solution was heated to 80° C. overnight. The mixture was cooled and EtOH (2 mL) was added. Then HOAc (0.15 mL) was added and most of the solvent was evaporated. MeOH was added and the solution was purified by preparative HPLC on an Xterra column using 50 mM ammonium bicarbonate, pH 10-acetonitrile as eluent.

General Synthetic Procedure for Hydrolysis of Tert-Butyl Carbamates (D)

The tert-butyl carbamate was dissolved in DCM (1 ml) and TFA (0.25 ml) was added. The mixtures were stirred at room temperature over night. The samples were concentrated and purified by preparative HPLC.

General Synthetic Procedure for Alkylation of Phenol by Alkylhalides (E)

The phenol (27 mg, 0.073 mmol) was dissolved in DMF (1 ml). K₂CO₃ (19 mg) and alkylhalides (0.0804 mmol) dissolved in CH₃CN (1 ml) were added. The mixtures were stirred at 80° C. for 2 days. The samples were concentrated and purified by preparative HPLC.

General Synthetic Procedure for Amide Formation (F)

The carboxylic acids (0.125 mmol) were weighed in 4-mL vials. A solution of the amine (0.1 mmol) in DMF (0.8 mL) was added followed by Et₃N (0.2 mmol). The coupling reagent TBTU (2-(1H-benzotriazol-1-yl)-1,1,3,3-tetramethyluroniumterafluoroborate, 0.12 mmol) was added to each vial. The solution was left at room temp overnight and then evaporated in vacuo. The residue was dissolved in DCM and passed through a short column (ca 4 mL) silica gel. The products were eluted with 0-3% MeOH/DCM.

General Synthetic Procedure for Urea Formation (G)

Tert-butyl 6-(3-aminophenyl)-1′H,4H-spiro[1,3-benzodioxine-2,4′-piperidine]-1′-carboxylate, (0.1 mmol) and Et₃N (0.4 mmol) were dissolved in DCM (0.5 mL) before a solution of triphosgene (0.06 mmol) in DCM (0.3 mL) was added slowly with cooling in ice bath. The mixture was left at room temp. for ½ h. Then the arylamines (0.1 mmol) dissolved in DCM (or CHCl₃-MeOH) was added. The solution was heated to 40° C. for 1.5 h and the left overnight at room temp. CHCl₃ (4 mL) and 0.2 M citric acid (2.5 mL) was added and the aqueous phase was removed. The organic phase was washed with brine and dried (Na₂SO₄). The residue was Boc-deprotected with DCM (0.8 mL) and TFA (0.2 mL) over ½ h at room temperature. The obtained crude product was purified by preparative HPLC on Xterra column with 50 mM NH₄HCO₃, pH 10/CH₃CN.

Intermediate Compounds

Intermediate 1:

### 1′-Carboethoxy-6-bromo-4H-spiro[1,3-benzodioxine-2,4′-piperidine]

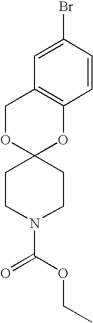

To a solution of 5-bromo-2-hydroxybenzyl alcohol (5.2 g, 26 mmol) and N-carboethoxy-4-piperidone (6.9 g, 34 mmol) in chloroform (70 mL) was added p-toluenesulphonic acid (0.5 g). The mixture was heated at reflux for 18 h using a Dean-Stark condenser to collect the water formed by the reaction. The solvent was removed in vacuo. The residue was dissolved in ethyl acetate (150 mL) and washed with a 2M sodium hydroxide solution (100 mL) and brine (100 mL). The organic layer was dried with magnesium sulfate and the solvent removed in vacuo. The residue was purified by column chromatography (silica gel, 20% EtOAc in petrol ether to 30% EtOAc in petrol ether) to give the title compound (5.7 g, 61% yield).

¹H NMR (400 MHz, CDCl₃) δ=1.22-1.27 (m, 3H, ethyl CH₃), 1.78-1.91 (m, 4H, 2×CH₂), 3.48-3.54 (m, 2H, CH₂), 3.59-3.65 (m, 2H, CH₂), 4.09-4.16 (m, 2H, ethyl CH₂), 4.78 (s, 2H, CH₂O), 6.71-6.74 (d, 1H, J=8.7 Hz, Harom), 7.08 (s, 1H, Harom) 7.23-7.25 (m, 1H, Harom).

Intermediate 2:

### 6-bromo-4H-spiro[1,3-benzodioxine-2,4′-piperidine]

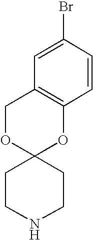

1′-Carboethoxy-6-bromo-4H-spiro[1,3-benzodioxine-2,4′-piperidine](Intermediate 1, 993 mg, 2.8 mmol) was dissolved in EtOH (30 mL) and 6M NaOH (8 mL). Solution was heated at reflux for 20 h. The mixture was evaporated in vacuo and the residue was partitioned between EtOAc and H₂O. The organic layer was dried with magnesium sulfate and solvent removed in vacuo to give the title compound as pale yellow oil (790 mg, 99% yield).

¹H NMR (400 MHz, CDCl₃) δ=1.60-1.73 (m, 4H, 2×CH₂), 1.77 (s, 1H, NH), 2.73-2.80 (m, 4H, 2×CH₂), 4.62 (s, 2H, CH₂O), 6.55-6.57 (d, J=8.7 Hz, 1H, Harom), 6.90-6.92 (m, 1H, Harom), 7.05-7.09 (m, 1H, Harom).

Intermediate 3:

### Ethyl 5-bromo-1′H-spiro[1,3-benzodioxole-2,4′-piperidine]-1′-carboxylate

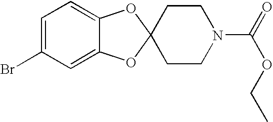

This compound was prepared by the general synthetic procedure A from 4-Bromocatechol (378 mg, 2 mmol), 1-carbethoxy-4-piperidone (445 mg, 2.6 mmol). Flash chromatography on silica gel with 15% EtOAc/hexane gave 531 mg product (78% yield).

HPLC 100%, R_{T} 2.71 (System A 10-97% MeCN over 3 min), LC-MS ES⁺ m/z 342. ¹H NMR (270 MHz, CDCl₃) δ 1.27 (t, 3H), 1.96 (m, 4H), 3.66 (m, 4H), 4.16 (q, 2H), 6.63 (d, 1H), 6.86-6.96 (m, 2H).

Intermediate 4:

### Ethyl 5-(3,4,5-trimethoxyphenyl)-1′H-spiro[1,3-benzodioxole-2,4′-piperidine]-1′-carboxylate

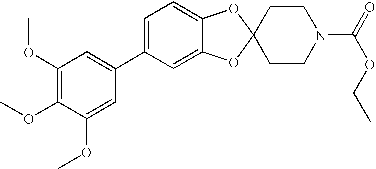

Ethyl 5-bromo-1′H-spiro[1,3-benzodioxole-2,4′-piperidine]-1′-carboxylate, and 3,4,5-trimethoxyboronic acid were reacted by the general procedure B2 yielding 144 mg crude product. Flash chromatography on silica gel with CHCl₃ as eluent gave the title compound (53 mg).

HPLC 95%, R_{T} 2.54 (System A 30-80% MeCN over 30 min), LC-MS ES⁺ m/z 430. ¹H NMR (270 MHz, CDCl₃) δ 1.28 (t, 3H), 2.01 (m, 4H), 3.70 (m, 4H), 3.84-3.94 (9H), 4.17 (q, 2H), 6.68 (s, 2H), 6.81 (d, 1H), 6.95-7.03 (m, 2H).

Intermediate 5:

### 6-Bromo-2′,3′,5′,6′-tetrahydro-4H-spiro[1,3-benzodioxine-2,4′-pyran]

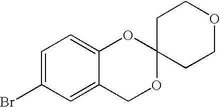

This compound was prepared according to general procedure A, from 5-bromo-2-hydroxybenzylalcohol (100 mg) and cyclohexanone (59 mg) to afford 45 mg of product. All material was used in the next step without further purification.

Intermediate 6:

### Ethyl 6-(2-hydroxyphenyl)-1′H,4H-spiro[1,3-benzodioxine-2,4′-piperidine]-1′-carboxylate

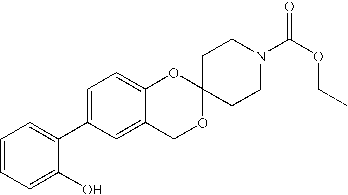

This compound was prepared in the same way as Example 5 from Intermediate 3 (600 mg), 2-(4,4,5,5-tetramethyl-1,3,2-dioxaborolan-2-yl)phenol (445 mg), NaHCO₃ (425 mg), PdCl₂dppf (69 mg) (general procedure A2). Purification by column chromatography using 3/4 CHCl₃:MeOH:heptan (4:1:4) and 1/4 heptan gave 413 mg.

HPLC 58%, R_{T}=2.298 (System B 10-97% MeCN over 3 min), MS m/z 397 (M+1)

Intermediate 7:

### Tert-butyl 6-(3-hydroxyphenyl)-1′H,4H-spiro[1,3-benzodioxine-2,4′-piperidine]-1′-carboxylate

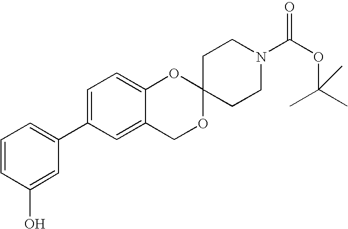

This compound was prepared according to general procedure B2 from tert-butyl 6-bromo-1′H,4H-spiro[1,3-benzodioxine-2,4′-piperidine]-1′-carboxylate (825 mg), 3-hydroxyphenylboronic acid (312 mg), NaHCO₃ (475 mg), PdCl₂dppf (77 mg). Purification by column chromatography using 3/4 CHCl₃:MeOH:heptane (4:1:4) and 1/4 heptane gave 572 mg.

HPLC 87% R_{T}=2.485(System B 10-97% MeCN over 3 min), MS m/z 298 (M-100)

Intermediate 8:

### Tert-butyl 6-(3-{2-[(2,6-dimethylphenyl)amino]-2-oxoethoxy}phenyl)-1′H,4H-spiro[1,3-benzodioxine-2,4′-piperidine]-1′-carboxylate

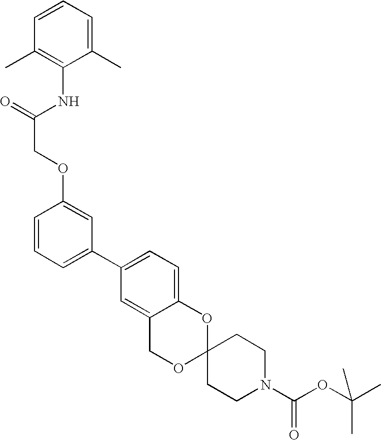

This compound was prepared according to general procedure E from 2-chloro-2′,6′-acetoxylidide (21 mg) and was used in the next step without further analysis.

HPLC 100%, R_{T} 2.948 (System C 10-97% MeCN over 3 min).

Intermediate 9:

### Tert-butyl 6-[3-(2-anilino-2-oxoethoxy)phenyl]-1′H,4H-spiro[1,3-benzodioxine-2,4′-piperidine]-1′-carboxylate

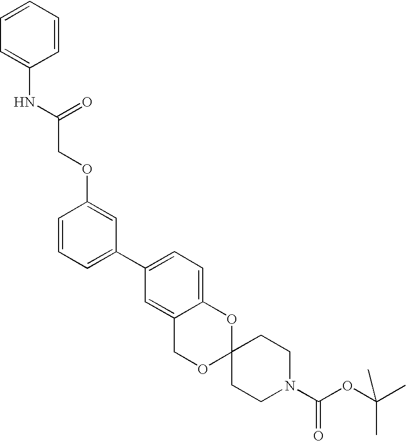

This compound was prepared according to general procedure E from 2-chloro-N-phenyl-acetamide (18 mg) and was used in the next step without further analysis.

HPLC 100%, R_{T} 2.929 (System C 10-97% MeCN over 3 min), MS (ES) m/z 431 (M−100).

Intermediate 10:

### Ethyl 6-[2-(2-amino-2-oxoethoxy)phenyl]-1′H,4H-spiro[1,3-benzodioxine-2,4′-piperidine]-1′-carboxylate

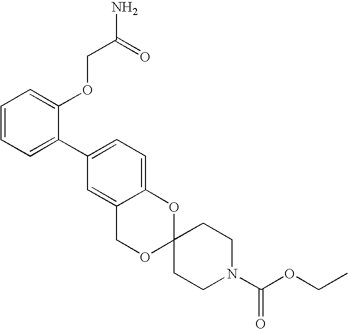

This compound was prepared according to general procedure E from 2-bromoacetamide (11 mg). All material was used for the next step (hydrolysis of the ethyl carbamate).

HPLC R_{T} 2.256 (System C 10-97% MeCN over 3 min).

Intermediate 11:

### Tert-butyl 6-[3-(2-oxo-2-{[2-(trifluoromethyl)phenyl]amino}ethoxy)phenyl]-1′H,4H-spiro[1,3-benzodioxine-2,4′-piperidine]-1′-carboxylate

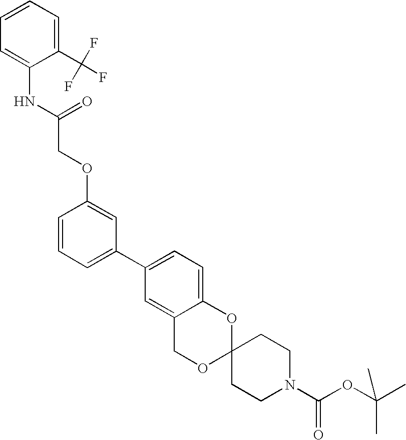

This compound was prepared according to general procedure E from N1-[3-(trifluoromethyl)phenyl]-2-chloroacetamide (25 mg). All material was used for the next step (hydrolysis of the t-butyl carbamate).

HPLC 100%, R_{T} 3.103 (System C 10-97% MeCN over 3 min).

Intermediate 12

### Tert-butyl 6-[3-(2-morpholin-4-ylethoxy)phenyl]-1′H,4H-spiro[1,3-benzodioxine-2,4′-piperidine]-1′-carboxylate

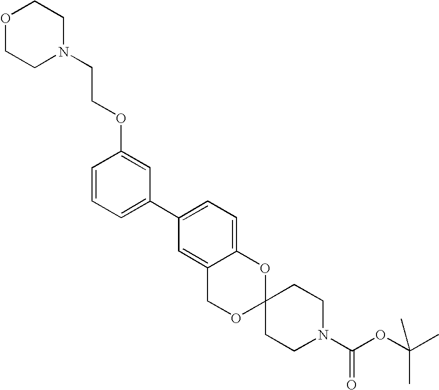

This compound was prepared according to general procedure E from N-(2-chloroethyl)morpholine hydrochloride (20 mg). All material was used for the next step (hydrolysis of the t-butyl carbamate).

HPLC 100%, R_{T} 2.853 (System C 10-97% MeCN over 3 min), MS (ES) m/z 511 (M+1).

Intermediate 13:

### Tert-butyl 6-bromo-1′H,4H-spiro[1,3-benzodioxine-2,4′-piperidine]-1′-carboxylate

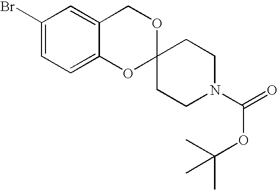

This compound was prepared by the general synthetic procedure A from 5-bromo-2-hydroxybenzyl alcohol and tert-butyl 4-oxopiperidine carboxylate. Yield: 1.21 μg, 42% of the title compound as an oil.

HPLC 96%, R_{T} 2.90 (System A 10-97% MeCN over 3 min), LC-MS ES⁺ m/z 185. ¹H NMR (270 MHz, CDCl₃) δ 1.45 (s, 9H), 1.86 (m, 4H), 3.37-3.69 (m, 4H), 6.74 (d, 1H), 7.10 (1H), 7.19-7.34 (m, 1H).

Intermediate 14:

### Tert-butyl 6-(3-aminophenyl)-1′H,4H-spiro[1,3-benzodioxine-2,4′-piperidine]-1′-carboxylate

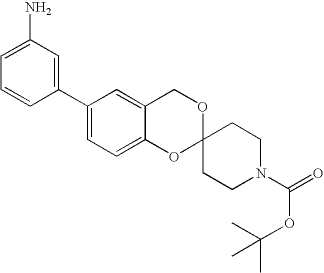

Tert-butyl 6-bromo-1′H,4H-spiro[1,3-benzodioxine-2,4′-piperidine]-1′-carboxylate (1.13 mg, 2.94 mmol), 3-aminophenylboronic acid hemisulfate (M=186, 1.64 g, 8.8 mmol) Pd(PPh₃)₄ (170 mg, 0.15 mmol) 1 M Na₂CO₃ (25 ml), EtOH (4.4 mL), DME (18 mL), were mixed and heated to 85° C. overnight. 50% EtOH was added and the mixture was filtered through Celite. Most of the filtrate was evaporated and DCM was added. The organic phase was washed with 2 M NaOH and brine. Yield 1.13 g crude product. Flash chromatography on silica gel with 0-1% MeOH/DCM and with 35% EtOAc/n-hexane gave 472 mg, 40%.

HPLC 100%, R_{T} 2.16 (System A 10-97% MeCN over 3 min), LC-MS ES⁺ m/z 397. ¹H NMR (270 MHz, CDCl₃) δ 1.46 (s, 9H), 1.77-2.02 (m, 4H), 3.41-3.78 (m, 6H), 4.89 (s, 2H), 6.83 (dd, 1H), 6.82 (m, 1H), 6.90 (d, 2H), 7.11-7.22 (m, 2H), 7.37 (dd, 1H).

Intermediate 15:

### Tert-butyl 6-(3-amino-4-methoxyphenyl)-1′H,4H-spiro[1,3-benzodioxine-2,4′-piperidine]-1′-carboxylate

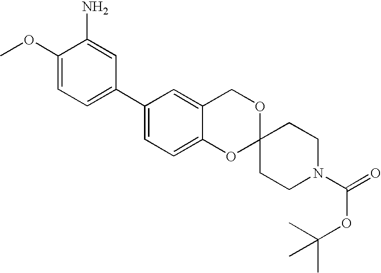

Tert-Butyl 6-bromo-1′H,4H-spiro[1,3-benzodioxine-2,4′-piperidine]-1′-carboxylate, (400 mg, 1.15 mmol), 3-amino-4-methoxyphenylboronic acid hydrochloride (350 mg, 1.72 mmol), Pd(PPh₃)₄ (66 mg, 0.0575 mmol), DME (6.9 mL), EtOH (1.6 mL), 1 M Na₂CO₃ (5 mL) were heated to 85° C. under nitrogen overnight. The reaction mixture was filtered through Celite and evaporated. The residue was dissolved in DCM and washed with 2 M NaOH. The organic phase was dried, filtered and evaporated. Flash-chromatography on silica gel with 20-35% EtOAc/n-hexane gave the product 200 mg as an oil.

HPLC 98%, R_{T} (System A 10-97% MeCN over 3 min), LC-MS ES⁺ m/z 427. ¹H NMR (400 MHz, CDCl₃) δ 1.46 (s, 9H), 1.80-1.98 (m, 4H), 3.43-3.54 (m, 2H), 3.55-3.67 (m, 2H), 3.87 (s, 3H), 4.88 (s, 2H), 6.83 (m, 1H), 6.87 (d, J=8.5 Hz, 1H), 6.92 (m, 1H), 6.96 (d, J=2.0 Hz, 1H), 7.11 (d, J=2.0 Hz, 1H), 7.32 (dd, J=8.5, 3.3 Hz, 1H).

Intermediate 16:

### 3-[I′-(Tert-butoxycarbonyl)-4H-spiro[1,3-benzodioxine-2,4′-piperidin]-6-yl]benzoic acid

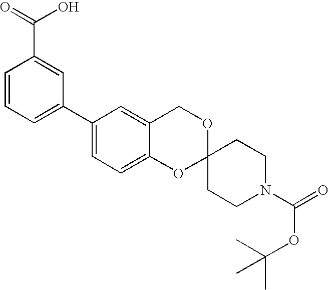

Tert-butyl 6-bromo-1′H,4H-spiro[1,3-benzodioxine-2,4′-piperidine]-′-carboxylate, (3.79 mmol) and 3-carboxybenzeneboronic acid (4.49 mmol) and Pd(PPh₃)₄ (0.19 mmol) were mixed with NaHCO₃ (9.52 mmol) and 80% dioxan (19 mL). Nitrogen was bubbled through, the tube was capped and then the mixture was stirred and heated to 80° C. for 5 h. The reaction mixture was filtered through Celite and washed with 50% EtOH. Most of the solvent was evaporated in vacuum and the residue was acidified with 12 mL 2 M citric acid. Extraction with DCM gave the crude product, which was purified by flash-chromatography on silica gel with 3-4% MeOH/CHCl₃. Got 0.57 g, yield 56%.

HPLC 100%, R_{T} 2.81 (System A 10-97% MeCN over 3 min), HPLC 98%, R_{T} 2.61 (System B 10-97% MeCN over 3 min), LC-MS ES⁺ m/z 227, 326. ¹H NMR (400 MHz, CDCl₃) δ 1.47 (s, 9H), 1.81-2.01 (m, 4H), 3.45-3.56 (m, 2H), 3.57-3.69 (m, 2H), 4.93 (s, 2H), 6.96 (d, J=8.5 Hz, 1H), 7.24 (d, J=2.3 Hz, 1H), 7.45 (dd, J=8.5, 2.3 Hz, 1H), 7.52 (t, J=7.8 Hz, 1H), 7.56 (m, 1H), 8.05 (ddd, J=7.9, 1.4, 1.3 Hz, 1H), 8.26 (t, J=1.6 Hz, 1H). ³C NMR (100.6 MHz, CDCl₃) δ 28.42, 60.35, 79.89, 98.32, 117.70, 119.99, 123.42, 127.07, 128.33, 128.53, 128.99, 129.82, 131.86, 132.65, 140.96, 150.63, 154.75, 171.37.

Intermediate 17:

### 5-Bromo-N-[2-(5-methoxy-1H-indol-3-yl)ethyl]nicotinamide

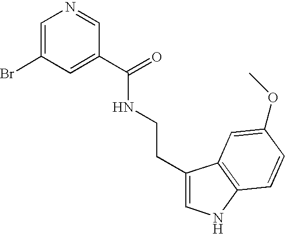

5-Bromonicotinic acid (1.1 mmol) and and 5-methoxytryptamine (0.5 mmol) were dissolved in DMF (5 mL). Triethylamine (2 eq.) was added and then TBTU (1.1 eq.). The mixture was stirred overnight at room temp and then was the solvent evaporated in vacuum. The residue was purified by flash-chromatography on RediSep columns with 40-50% EtOAc/toluene. This gave 91 mg of the product. HPLC 91%, R_{T} 1.96 (System A 10-97% MeCN over 3 min), LC-MS ES⁺ m/z 376.

Intermediate 19:

### N-(5-bromopyridin-3-yl)-2-(3,4,5-trimethoxyphenyl)acetamide

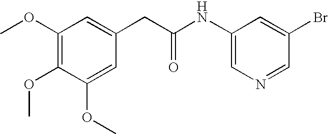

This compound was prepared using a similar procedure as for Intermediate 17 to afford 192 mg of the product, (87% purity).

HPLC 87%, R_{T} 1.89 (System A 10-97% MeCN over 3 min), LC-MS ES⁺ m/z 381. ¹H NMR (400 MHz, CDCl₃) δ 3.71 (s, 2H), 3.85 (s, 3H), 3.86 (s, 6H), 6.53 (s, 2H), 8.37 (d, J=2.0 Hz, 1H), 8.45 (d, J=2.0 Hz, 1H), 8.51 (m, 1H).

Intermediate 20:

### N-(5-bromopyridin-3-yl)-2-(5-methoxy-1H-indol-3-yl)acetamide

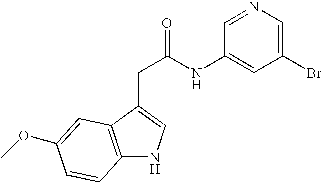

This compound was prepared using a similar procedure as for Intermediate 17 to afford 152 mg of the product, (72% purity)

HPLC 72%, R_{T} 1.90 (System A 10-97% MeCN over 3 min), LC-MS ES⁺ m/z 360.

Intermediate 21:

### 6-bromo-N-[2-(5-methoxy-1H-indol-3-yl)ethyl]pyridine-2-carboxamide

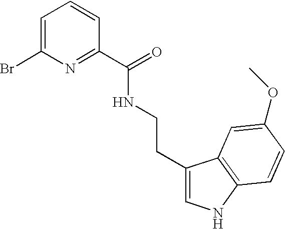

This compound was prepared using a similar procedure as for Intermediate 17 to afford 116 mg of the product.

HPLC 100%, R_{T} 2.22 (System A 10-97% MeCN over 3 min), LC-MS ES⁺ m/z 374. ¹H NMR (400 MHz, CDCl₃) δ 3.07 (t, J=6.9 Hz, 2H), 3.73-3.92 (m, 2H), 3.84 (s, 3H), 6.86 (dd, J=8.8, 2.3 Hz, 1H), 7.05-7.10 (m, 2H), 7.58 (dd, J=8.0, 1 Hz, 1H), 7.70 (t, J=7.7 Hz, 1H), 7.94 (m, 1H), 8.16 (dd, J=7.5, 1 Hz, 1H).

Intermediate 22:

### Tert-butyl 6-(3-{[(3,4,5-trimethoxyphenyl)acetyl]amino}phenyl)-1′H,4H-spiro[1,3-benzodioxine-2,4′-piperidine]-1′-carboxylate

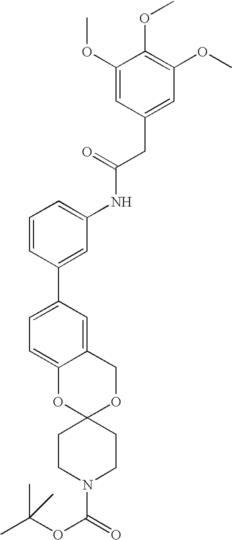

3,4,5-trimethoxyphenylacetic acid (1.0 g, 4.4 mmol), 3-(4,4,5,5-Tetramethyl-1,3,2-dioxaborolan-2-yl)aniline (1.25 g, 5.3 mmol) and triethylamine (1.3 g, 13.2 mmol) were dissolved in THF (30 mL) and HOBT (0.90 g, 6.6 mmol) and EDC (1.27 g, 6.6 mmol) were added and the mixture was stirred overnight at room temperature. The mixture was evaporated and dissolved in dichloromethane and purified by flash chromatography using 1% MeOH to 5% MeOH in dichloromethane.

NMR shows mostly the pinacol ester but also the boronic acid. Yield 0.9 g as a white powder. This mixture (0.160 g, 0.37 mmol), tert-butyl 6-bromo-1′H,4H-spiro[1,3-benzodioxine-2,4′-piperidine]-1′-carboxylat (0.100 g, 0.26 mmol), NaHCO₃ (0.100 g, 1.2 mmol), water (1 mL) and tetrakis palladium (0.020 g, 0.017 mmol) were dissolved in DME (4 mL1) and heated in the microwave at 130° C. for 20 minutes. The mixture was evaporated and partitioned between water and dichloromethane. The organic phase was dried (MgSO₄) and evaporated. The crude product was purified by flash chromatography using hexane/ethyl acetate 1:1 as the eluent. Yield 100 mg (64%). White powder.

HPLC 95% Rt=2.72 min (system A. 10-97% MeCN over 3 minutes). HPLC 98% Rt=2.80 min (system B. 10-97% MeCN over 3 minutes). MS (electronspray; [M-100]+) m/z 505.4. ¹H NMR (400 MHz, CHLOROFORM-D) δ ppm 1.46 (s, 9H) 1.80-1.98 (m, 4H) 3.44-3.65 (m, 4H) 3.68 (s, 2H) 3.83-3.88 (m, J=3.51 Hz, 9H) 4.89 (s, 2H) 6.53 (s, 2H) 6.90 (d, J=8.53 Hz, 1H) 7.14-7.24 (m, 3H) 7.31-7.39 (m, 3H) 7.64 (s, 1H).

Intermediate 23:

### 5-Bromo-2-fluoro-N-[2-(5-methoxy-1H-indol-3-yl)-ethyl]-benzamide

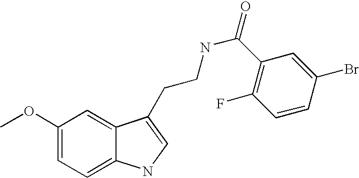

This compound was prepared using a similar procedure as for Intermediate 17 to afford 125 mg of the product.

HPLC 92%, R_{T} 2.27 (System A 10-97% MeCN over 3 min), LC-MS ES⁺ m/z 392.

### EXAMPLES

### Example 1

### 6-[quinolin-5-yl]-4H-spiro[1,3-benzodioxine-2,4′-piperidine]

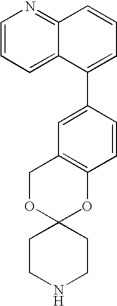

To a solution of 6-bromo-4H-spiro[1,3-benzodioxine-2,4′-piperidine](Intermediate 2) (103 mg, 0.4 mmol), quinoline-5-boronic acid (132 mg, 0.8 mmol), NaHCO₃ (91 mg, 1.1 mmol) in 4 mL of degassed DME:H₂O (3:1) was added Pd(dppf)Cl₂ (33 mg, 0.064 mmol). Mixture was heated at 80° C. under nitrogen for 16 h. Reaction mixture was allowed to cool down to room temperature and partitioned between EtOAc and H₂O. Organic layer was dried with magnesium sulfate and solvent removed in vacuo to give a crude which was purified by combiflash (dichloromethane/7N methanolic NH₃) followed by biotage using a gradient of dichloromethane/7N methanolic NH₃ (88.5:1.5 to 97:3) to give the title compound as a brown solid (28.8 mg, 24% yield).

¹H NMR (400 MHz, CDCl₃) δ=1.94-2.07 (m, 4H, 2×CH₂), 3.03-3.07 (m, 4H, 2×CH₂), 4.94 (s, 2H, CH₂O), 7.00-7.02 (m, 1H, Harom), 7.07 (s, 1H, Harom), 7.26-7.28 (m, 1H, Harom), 7.35-7.38 (m, 1H, Harom), 7.46-7.47 (m, 1H, Harom), 7.72-7.76 (m, 1H, Harom), 8.09-8.12 (m, 1H, Harom), 8.24-8.27 (m, 1H, Harom), 8.92-8.93 (m, 1H, Harom). HPLC 99.86%, R_{T}=2.80 mins. MS (ES⁺) m/z 333 (M+H).

### Example 2

### 6-[3,4,5-trimethoxyphenyl]-4H-spiro[1,3-benzodioxine-2,4′-piperidine]

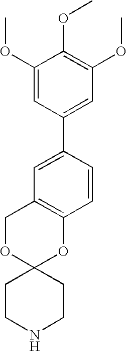

Same protocol as for Example 1 to give the title product (15.7 mg, 12% yield).

¹H NMR (400 MHz, CDCl₃) δ=1.85-2.00 (m, 4H, 2×CH₂), 2.96-3.00 (m, 4H, 2×CH₂), 3.88 (s, 3H, —OCH₃), 3.92 (s, 6H, 2× —OCH₃), 4.92 (s, 2H, —OCH₂), 6.70 (s, 2H, Harom), 6.92-6.94 (m, 1H, Harom), 7.14 (s, 1H, Harom), 7.35-7.38 (m, 1H, Harom). HPLC 94.60% R_{T}=2.17 mins. MS (AP⁺) m/z 372 (M+H).

### Example 3

### 6-[2,4-dimethoxyphenyl]-4H-spiro[1,3-benzodioxine-2,4′-piperidine]

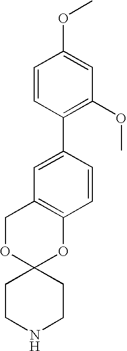

Same protocol as for Example 1 to give the title compound (23 mg, 17% yield).

¹H NMR (400 MHz, CDCl₃) δ=1.72 (br s, 1H, NH), 1.84-1.99 (m, 4H, 2×CH₂), 2.95-3.04 (m, 4H, 2×CH₂), 3.79 (s, 3H, —OCH₃), 3.84 (s, 3H, —OCH₃), 4.72 (s, 2H, —OCH₂), 6.53-6.56 (m, 2H, Harom), 6.88-6.90 (d, 1H, J=8.4 Hz, Harom), 7.10 (s, 1H, Harom), 7.18-7.20 (m, 1H, Harom), 7.29-7.32 (m, 1H, Harom). HPLC 95.95% R_{T}=3.24 mins. MS (AP⁺) m/z 342 (M+H).

### Example 4

### 5-(3,4,5-trimethoxyphenyl)spiro[1,3-benzodioxole-2,4′-piperidine]

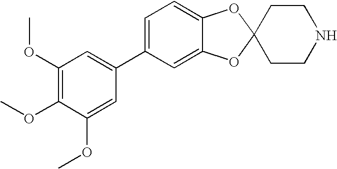

This compound was prepared from the ethylcarbamate (53 mg, Intermediate 4) by the general procedure C to afford 25 mg of the title compound.

HPLC 100%, R_{T} 1.76 (System A 10-97% MeCN over 3.0 min), LC-MS ES⁺ m/z 358. ¹H NMR (270 MHz, MeOH-d4-CDCl₃) δ 2.04 (m, 4H), 3.12 (m, 4H), 3.71 (s, 3H), 3.76 (s, 6H), 6.55 (s, 2H), 6.65-6.73 (d, 1H), 6.81-6.91 (m, 2H).

### Example 5

### 5-(2′,3′,5′,6′-Tetrahydro-4H-spiro[1,3-benzodioxine-2,4′-pyran]-6-yl)quinoline

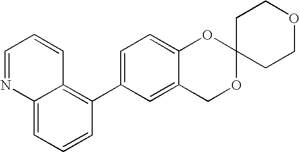

To 6-Bromo-2′,3′,5′,6′-tetrahydro-4H-spiro[1,3-benzodioxine-2,4′-pyran](Intermediate 5; 45 mg), guinoline-5-boronic acid (33 mg), PdCl₂dppf (6.4 mg) and NaHCO₃ (40 mg) was added 80% aq. EtOH (2 mL). The mixture was stirred at 80° C. over night. The mixture was suspended in ethanol and filtered through celite. The solvent was removed in vacuo. Purification by preparative HPLC gave 5 mg of the title compound.

HPLC 100%, R_{T} 1.504 (System B 10-97% MeCN over 3 min)

### Example 6

### 6-(3,4,5-trimethoxyphenyl)-4H-spiro[1,3-benzodioxine-2,3′-piperidine],

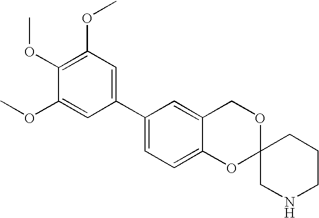

1-Boc-3-piperidone (500 mg, 2.50 mmol), 5-bromo-2-hydroxybenzyl alcohol (406 mg, 2.0 mmol), TsOH monohydrate (38 mg, 0.2 mmol) and CHCl₃ (10 mL) was heated to reflux with a Dean-Stark condensor overnight. The solvent was evaporated. A Suzuki reaction was run on the crude product.

3,4,5-Trimethoxybenzeneboronic acid (509 mg, 2.4 mmol), PPh₃ (79 mg, 0.3 mmol), NaHCO₃ (504 mg, 6 mmol) and 80% EtOH (10 mL) was added. Then Pd(OAc)₂ (22 mg, 0.1 mmol) was added. Heated to 70° C. overnight. The mixture was filtered through Celite, the filtrate was evaporated and the residue mixed with DCM. Washed with 5% NaHCO₃ and brine. Flash chromatography gave a fraction that contained a peak with the expected m/z-100. This fraction was flashed again with 20-40% EtOAc/hexane to give 109 mg of the the title compound, ca 87% purity (12% yield). This material was treated with TFA-DCM 1:4 (2 mL) for 30 min. The solution was evaporated in vacuum to give 138 mg crude product. Preparative HPLC on Xterra column with 26-56% CH₃CN/NH₄HCO₃-pH 10 gave 30 mg (salt free).

HPLC 100%, R_{T} 1.74 (System A 10-97% MeCN over 3 min), LC-MS ES⁺ m/z 372. ¹H NMR (270 MHz, CDCl₃) δ 1.68 (bs, 2H), 1.96 (m, 2H), 2.83 (m, 2H), 2.97 (bs, 2H), 3.86 (s, 3H), 3.90 (s, 6H), 4.90 (d, 1H), 4.91 (d, 1H), 6.68 (s, 2H), 6.92 (d, 1H), 7.12 (s, 1H), 7.35 (dd, 1H).

### Example 7

### N-(2,6-dimethylphenyl)-2-[3-(4H-spiro[1,3-benzodioxine-2,4′-piperidin]-6-yl)phenoxy]acetamide

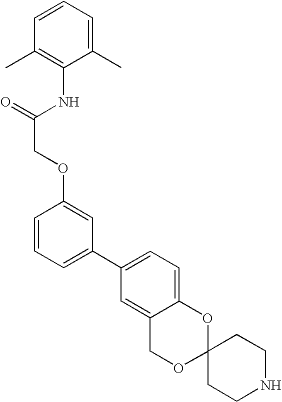

This compound was prepared from the tert-butylcarbamate (Intermediate 8) by the general procedure D to afford 2.3 mg.

HPLC 100%, R_{T}=1,856 min, MS (ES) m/z 459 (M). ¹H-NMR (270 MHz, MeOD) δ=1.87-1.97 (m, 4H), 2.10-2.18 (m, 6H), 2.88-2.98 (m, 4H), 4.79-4.80 (s, 2H), 4.92-4.94 (s, 2H), 6.88-6.93 (d, 1H, J=8.41 Hz), 6.99-7.10 (m, 4H), 7.20-7.48 (m, 5H)

### Example 8

### N-phenyl-2-[3-(4H-spiro[1,3-benzodioxine-2,4′-piperidin]-6-yl)phenoxy]acetamide

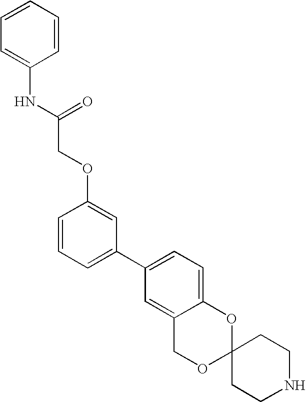

This compound was prepared from Intermediate 9 according to general procedure D to afford 2.4 mg.

HPLC 100%, R_{T}=1,849 min, MS (ES) m/z 431 (M)

### Example 9

### 2-[2-(4H-spiro[1,3-benzodioxine-2,4′-piperidin]-6-yl)phenoxy]acetamide

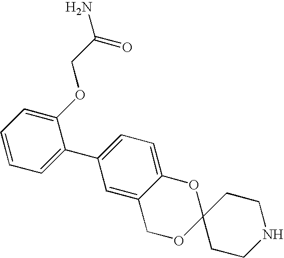

This compound was prepared from the ethylcarbamate (Intermediate 10) by the general procedure D to afford 0.3 mg. HPLC 100%, R_{T}=1.522 min, MS (ES) m/z 356 (M+1)

¹H-NMR (270 MHz, MeOD) δ=1.92-1.99 (m, 4H), 2.97-3.05 (m, 4H), 4.32-4.34 (s, 2H), 4.9-4.93 (s, 2H), 6.81-6.86 (d, 1H, J=8.17 Hz), 6.90-6.99 (m, 2H), 7.16-7.27 (m, 2H), 7.35-7.42 (m, 2H)

### Example 10

### 2-[3-(4H-spiro[1,3-benzodioxine-2,4′-piperidin]-6-yl)phenoxy]-N-[2-(trifluoromethyl)phenyl]acetamide

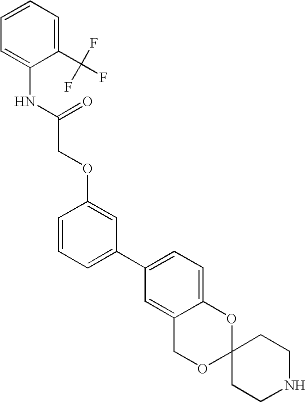

This compound was prepared from the tert-butylcarbamate (Intermediate 11) by the general procedure D to afford 1.4 mg.

HPLC 100%, R_{T}=1,849 min, MS (ES) m/z 431 (M). ¹H-NMR (270 MHz, MeOD) δ=1.86-1.94 (m, 4H), 2.88-2.96 (m, 4H), 4.72-4.76 (s, 2H), 4.90-4.93 (s, 2H), 6.85-6.92 (d, 1H, J=8.66 Hz), 6.96-7.02 (m, 1H), 7.18-7.56 (m, 7H), 7.83-7.89 (m, 1H), 8.05-8.10 (m, 1H)

### Example 11

### 6-[3-(2-morpholin-4-ylethoxy)phenyl]-4H-spiro[1,3-benzodioxine-2,4′-piperidine]

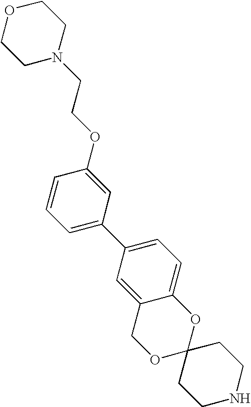

This compound was prepared from the tert-butylcarbamate (Intermediate 12) by the general procedure D to afford 0.4 mg.

HPLC 100%, R_{T}=1.242 min, MS (ES) m/z 411 (M+1)

### Example 12

### 2-(5-methoxy-1H-indol-3-yl)-N-[3-(4H-spiro[1,3-benzodioxine-2,4′-piperidin]-6-yl)phenyl]acetamide

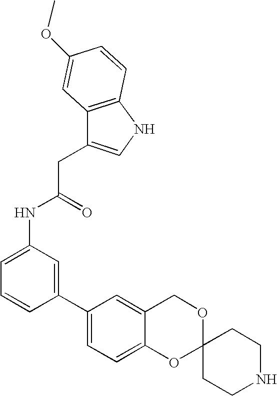

This compound was prepared from tert-butyl 6-(3-aminophenyl)-1′H,4H-spiro[1,3-benzodioxine-2,4′-piperidine]-1′-carboxylate by the general synthetic procedure F. The amides were further purified by preparative HPLC with an Xterra column (0.05 M NH₄HCO₃, pH 10/acetonitrile, 25 mL/min). Boc-deprotection was made with DCM (0.8 mL) and TFA (0.2 mL) for ca 35 min. Evaporation of the solvent and preparative HPLC (Xterra) gave 3.3 mg of the title compound.

HPLC 100%, R_{T} 1.78 (System A 10-97% MeCN over 3 min), LC-MS ES⁺ m/z 484. ¹H NMR (270 MHz, CDCl₃) δ 1.91 (m 4H), 2.98 (m, 4H), 3.82 (s, 3H), 3.88 (s, 2H), 4.87 (s, 2H), 6.82-6.97 (m, 2H), 7.02 (d, 1H), 7.13 (s, 1H), 7.17-7.29 (m), 7.32 (d, 1H), 7.45 (s, 1H), 7.57 (s, 1H), 8.20 (s, 1H).

### Example 13

### N-[3-(4H-spiro[1,3-benzodioxine-2,4′-piperidin]-6-yl)phenyl]-2-(3,4,5-trimethoxyphenyl)acetamide

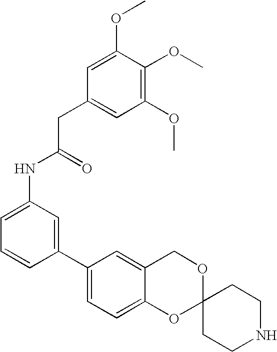

Prepared using a similar procedure as for Example 12 to afford 2.9 mg.

HPLC 100%, R_{T} 1.78 (System A 10-97% MeCN over 3 min), LC-MS ES⁺ m/z 505. ¹H NMR (270 MHz, CDCl₃) δ 1.93 (m, 4H), 3.01 (m, 4H), 3.69 (s, 2H), 3.86, 3.87 (s, 9H), 4.89 (s, 2H), 6.54 (s, 2H), 6.91 (d, 1H), 7.17 (s, 2H), 7.30-7.41 (m, 3H), 7.63 (s, 1H).

### Example 14

### 2-(4-fluorophenoxy)-N-[3-(4H-spiro[1,3-benzodioxine-2,4′-piperidin]-6-yl)phenyl]acetamide

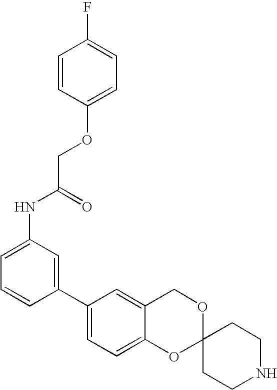

Prepared using a similar procedure as for Example 12 to afford 4.9 mg.

HPLC 100%, R_{T} 2.05 (System A 10-97% MeCN over 3 min), LC-MS ES⁺ m/z 449. ¹H NMR (270 MHz, CDCl₃) δ 1.92 (m, 4H), 2.99 (m, 4H), 4.58 (s, 2H), 4.91 (s, 2H), 6.89-7.09 (m, 5H), 7.20 (d, 1H), 7.28-7.44 (m, 3H), 7.49 (d, 1H), 7.78 (s, 1H), 8.28 (s, 1H).

### Example 15

### 1-(4H-spiro[1,3-benzodioxine-2,4′-piperidin]-6-yl)isoquinolin-3-amine

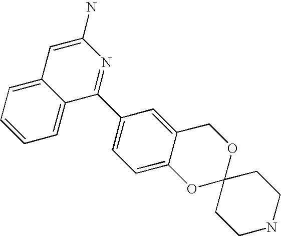

To a solution of ethyl 6-bromo-1′H,4H-spiro[1,3-benzodioxine-2,4′-piperidine]1′-carboxylate (48 mg, 0.281 mmol), Pd(OAc)₂ (1.5 mg, 0.007 mmol) and 2-(dicyclohexylphosphino)biphenyl (9.4 mg, 0.026 mmol) in 2 mL dioxane, TEA (0.122 mL, 1.12 mmol) was added. Pinacolborane (58 μL, 0.404 mmol) was then added dropwise and the mixture was heated at 80 deg for 2 h. Water (0.2 mL), Ba(OH)2×8H2O 267 mg, 0.842 mmol) and 1-bromoisoquinolin-3-amine (36 mg, 0.162 mmol) in dioxane (1 mL) was added and the resulting mixture was heated at 100 deg for 3 h. The mixture was filtered through celite and the solvent was removed. Purification by preparative HPLC gave 7 mg of ethyl 6-(3-aminoisoquinolinyl)-1′H,4H-spiro[1,3-benzodioxine-2,4′-piperidine]-1′-carboxylate.

The product was dissolved in 1 mL EtOH and 0.3 mL 6M NaOH was added. The mixture was heated at 100° C. over night, after which the solvent was removed. The residue was dissolved in DCM/MeOH 9: 1×0.4% NH₃(aq) and was run through a silica plug using the same solvent mixture as eluent. The solvent was removed and 1.2 mg (3%) of the title compound was obtained.

HPLC 93 R_{T} 1.35 min System A 10-97% MeCN over 3 min, LC-MS ES⁺ m/z 348

### Example 16

### N-[3-(4H-spiro[1,3-benzodioxine-2,4′-piperidin]-6-yl)phenyl]pyrazine-2-carboxamide

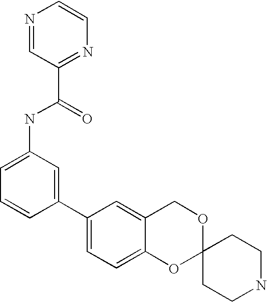

This compound was prepared using a similar procedure as for Example 12 to afford 25 mg of the product.

HPLC 99%, R_{T} 1.73 (System A 10-97% MeCN over 3 min), LC-MS ES⁺ m/z 403. ¹H NMR (270 MHz, MeOH-d4) δ1.83-1.96 (m, 4H), 2.92 (m, 4H), 6.88 (d, 1H), 7.24-7.48 (m, 4H), 7.63-7.772 (m, 1H), 8.00 (m, 1H), 8.71 (d, 1H), 8.79 (d, 1H), 9.32 (s, 1H).

### Example 17

### 2-(3,4-dimethoxyphenyl)-N-[3-(4H-spiro[1,3-benzodioxine-2,4′-piperidin]-6-yl)phenyl]acetamide

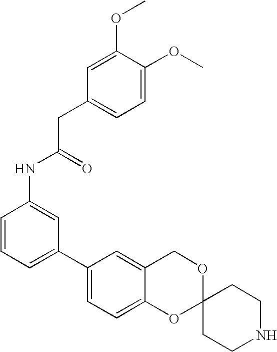

This compound was prepared using a similar procedure as for Example 12 to afford 17 mg of the product.

HPLC 100%, R_{T} 1.76 (System A 10-97% MeCN over 3 min), LC-MS ES⁺ m/z 475. ¹H NMR (270 MHz, MeOD) δ 1.90 (m, 4H), 2.92 (m, 4H), 3.62 (s, 2H), 3.80 (s, 3H), 3.83 (s, 3H), 4.90 (obscured by solvent peak), 6.84-6.93 (m, 3H), 6.98 (s, 1H), 7.24-7.37 (m, 3H), 7.37-7.48 (m, 2H), 7.80 (s, 1H).

### Example 18

### N-[2-(3,4-dimethoxyphenyl)ethyl]-3-(4H-spiro[1,3-benzodioxine-2,4′-piperidin]-6-yl)benzamide

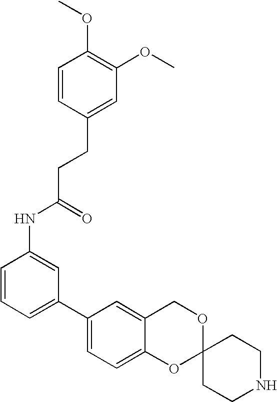

This compound was prepared using a similar procedure as for Example 12 to afford 36 mg of the product.

HPLC 100%, Rₜ 1.529 (10-97% MeCN over 3 min), MS (ES) m/z 489 (M+1). ¹H-NMR (270 MHz, MeOD) δ 1.83-1.95 (m, 4H), 2.83-2.88 (m, 2H), 2.89-2.97 (m, 4H), 3.53-3.64 (m, 2H), 3.75-3.79 (d, 6H, J=5.69 Hz), 4.92-4.94 (s, 2H), 6.77-6.96 (m, 4H), 7.29-7.35 (m, 1H), 7.42-7.51 (m, 2H), 7.65-7.75 (m, 2H), 7.88-7.94 (m, 1H).

### Example 19

### 3-(4H-spiro[1,3-benzodioxine-2,4′-piperidin]-6-yl)-N-(3,4,5-trimethoxybenzyl)benzamide

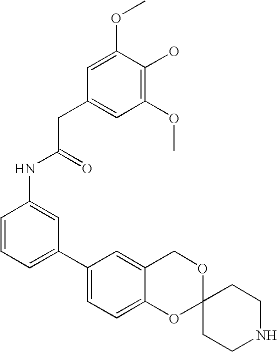

This compound was prepared using a similar procedure as for Example 12 to afford 49 mg of the product.

HPLC 100%, Rt 1.629 (10-97% MeCN over 3 min), MS (ES) m/z 505 (M+1). ¹H-NMR (270 MHz, MeOD) δ 1.86-1.93 (m, 4H), 2.88-2.96 (m, 4H), 3.68-3.75 (m, 3H), 3.81-3.84 (m, 6H), 4.49-4.54 (m, 2H), 4.91-4.94 (m, 2H) 6.67-6.71 (m, 2H), 6.88-6.93 (d, 1H, J=8.66 Hz), 7.33-7.38 (m, 1H), 7.45-7.54 (m, 2H), 7.71-7.81 (m, 2H), 8.03-8.12 (m, 1H).

### Example 20

### 2-(2,3-dimethoxyphenyl)-N-[3-(4H-spiro[1,3-benzodioxine-2,4′-piperidin]-6-yl)phenyl]acetamide

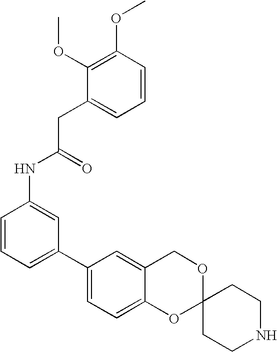

This compound was prepared using a similar procedure as for Example 12 to afford 20 mg of the product.

HPLC 100%, R_{T} 1.94 (System A 10-97% MeCN over 3 min), LC-MS ES⁺ m/z 475. ¹H NMR (500 MHz, CDCl₃) δ 1.81-1.97 (m, 4H), 2.90-3.03 (m, 4H), 3.71 (s, 2H), 3.88 (s, 3H), 3.93 (s, 3H), 4.88 (s, 2H), 6.86-6.95 (m, 3H), 7.05 (t, J=8 Hz, 1H), 7.15 (d, J=2 Hz, 1H), 7.19 (d, J=7.9 Hz, 1H), 7.28 (t, J=7.9 Hz, 1H), 7.30-7.38 (m, 2H), 7.67 (bs, 1H), 7.95 (bs, 1H).

### Example 21

### 2-(2,4-dimethoxyphenyl)-N-[3-(4H-spiro[1,3-benzodioxine-2,4′-piperidin]-6-yl)phenyl]acetamide

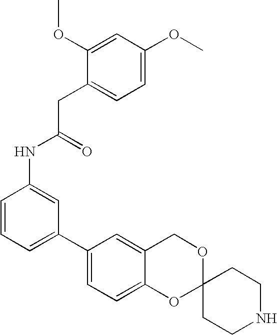

This compound was prepared using a similar procedure as for Example 12 to afford 14 mg of the product.

HPLC 100%, R_{T} 1.97 (System A 10-97% MeCN over 3 min), LC-MS ES⁺ m/z 475. ¹H NMR (500 MHz, CDCl₃) δ 1.81-1.96 (m, 4H), 2.91-3.02 (m, 4H), 3.64 (s, 2H), 3.81 (s, 3H), 3.88 (s, 3H), 4.88 (s, 2H), 6.48-6.54 (m, 2H), 6.89 (d, J=8.5 Hz, 1H), 7.16 (d, J=1.9 Hz, 1H), 7.18-7.22 (m, 2H), 7.25-7.31 (m, 2H), 7.36 (dd, J=8.5, 2.2 Hz, 1H), 7.59 (s, 1H), 7.67 (s, 1H).

### Example 22

### 3-(1H-indol-3-yl)-N-[3-(4H-spiro[1,3-benzodioxine-2,4′-piperidin]-6-yl)phenyl]propanamide

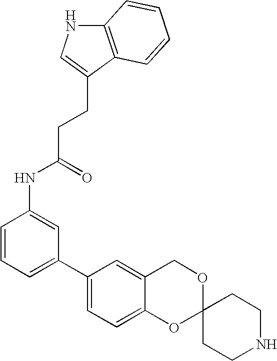

This compound was prepared using a similar procedure as for Example 12 to afford 21 mg of the product.

HPLC 100%, R_{T} 1.96 (System A 10-97% MeCN over 3 min), LC-MS ES⁺ m/z 468. ¹H NMR (500 MHz, CDCl₃) δ 1.81-1.97 (m, 4H), 2.77 (t, J=7.2 Hz, 2H), 2.91-3.02 (m, 4H), 3.22 (t, J=7.1 Hz, 2H), 4.89 (s, 2H), 6.91 (d, J=8.5 Hz, 1H), 7.02-7.07 (m, 2H), 7.11-7.16 (m, 2H), 7.18-7.24 (m, 3H), 7.28 (d, J=7.9 Hz, 1H), 7.36 (m, 2H), 7.59 (s, 1H), 7.64 (d, 1H), 7.98 (s, 1H).

### Example 23

### 2-(5-methoxy-2-methyl-1H-indol-3-yl)-N-[3-(4H-spiro[1,3-benzodioxine-2,4′-piperidin]-6-yl)phenyl]acetamide

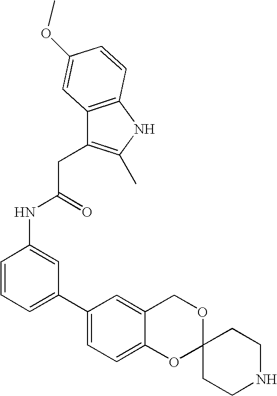

This compound was prepared using a similar procedure as for Example 12 to afford 14 mg of the product.

HPLC 100%, R_{T} 1.91 (System A 10-97% MeCN over 3 min), LC-MS ES⁺ m/z 498. ¹H NMR (500 MHz, CDCl₃) δ 1.79-1.97 (m, 4H), 2.43 (s, 3H), 2.91-3.01 (m, 4H), 3.79 (s, 2H), 3.81 (s, 3H), 4.87 (s, 2H), 6.84 (dd, J=8.6, 2.4 Hz, 1H), 6.88 (d, J=8.5 Hz, 1H), 6.94 (d, J=2.2 Hz, 1H), 7.13 (d, J=1.9 Hz, 1H), 7.18-7.24 (m, 3H), 7.30-7.35 (m, 1H), 7.41 (s, 1H), 7.54 (s, 1H), 7.94 (s, 1H).

### Example 24

### 2-(2-methyl-1H-indol-3-yl)-N-[3-(4H-spiro[1,3-benzodioxine-2,4′-piperidin]-6-yl)phenyl]acetamide

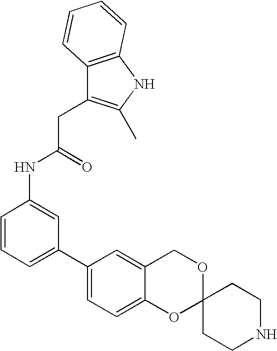

This compound was prepared using a similar procedure as for Example 12 to afford 25 mg of the product.

HPLC 100%, R_{T} 1.96 (System A 10-97% MeCN over 3 min), LC-MS ES⁺ m/z 468. ¹H NMR (500 MHz, CDCl₃) δ 1.81-1.96 (m, 4H), 2.45 (s, 3H), 2.92-3.02 (m, 4H), 3.83 (s, 2H), 4.87 (s, 2H), 6.88 (d, J=8.5 Hz, 1H), 7.11-7.17 (m, 2H), 7.17.7.22 (m, 3H), 7.30-7.37 (m, 2H), 7.38 (s, 1H), 7.49-7.56 (m, 2H), 8.06 (s, 1H).

### Example 25

### 2-(7-methyl-1H-indol-3-yl)-N-[3-(4H-spiro[1,3-benzodioxine-2,4′-piperidin]-6-yl)phenyl]acetamide

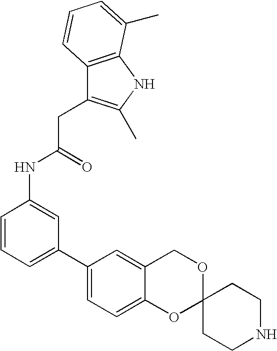

This compound was prepared using a similar procedure as for Example 12 to afford 19 mg of the product.

HPLC 100%, R_{T} 2.00 (System A 10-97% MeCN over 3 min), LC-MS ES⁺ m/z 468. ¹H NMR (500 MHz, CDCl₃) δ 1.80-1.97 (m, 4H), 2.53 (s, 3H), 2.90-3.03 (m, 4H), 3.90 (s, 2H), 4.87 (s, 2H), 6.88 (d, J=8.5 Hz, 1H), 7.05-7.15 (m, 3H), 7.17-7.23 (m, 2H), 7.32 (dd, J=8.3, 1.7 Hz, 1H), 7.43 (s, 1H), 7.48 (d, J=7.9 Hz, 1H), 7.57 (s, 1H), 8.20 (s, 1H).

### Example 26

### N-[3-(4H-spiro[1,3-benzodioxine-2,4′-piperidin]-6-yl)phenyl]-3-(3,4,5-trimethoxyphenyl)propanamide

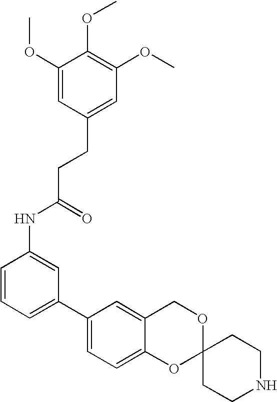

This compound was prepared using a similar procedure as for Example 12 to afford 30 mg of the product.

HPLC 100%, R_{T} 1.88 (System A 10-97% MeCN over 3 min), LC-MS ES⁺ m/z 519. ¹H NMR (500 MHz, CDCl₃) δ 1.82-1.98 (m, 4H), 2.65 (m, 2H), 2.93-3.03 (m, 6H), 3.79 (s, 6H), 3.81 (s, 3H), 4.90 (s, 2H), 6.45 (s, 2H), 6.91 (d, J=8.5 Hz, 1H), 7.03 (s, 1H), 7.17 (s, 1H), 7.27-7.35 (m, 2H), 7.38 (dd, J=8.5, 1.9 Hz, 1H), 7.65 (s, 1H).

### Example 27

### 3-phenoxy-N-[3-(4H-spiro[1,3-benzodioxine-2,4′-piperidin]-6-yl)phenyl]propanamide

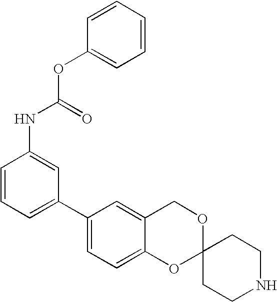

This compound was prepared using a similar procedure as for Example 12 to afford 10 mg of the product.

HPLC 100%, R_{T} 2.01 (System A 10-97% MeCN over 3 min), LC-MS ES⁺ m/z 445. ¹H NMR (500 MHz, CDCl₃) δ 1.81-1.98 (m, 4H), 2.85 (t, J=5.8 Hz, 2H), 2.91-3.04 (m, 4H), 4.36 (t, J=5.8 Hz, 2H), 4.89 (s, 2H), 6.91 (d, J=8.5 Hz, 1H), 7.18 (d, J=1.9 Hz, 1H), 7.28-7.41 (m, 5H), 7.74-7.84 (m, 2H).

### Example 28

### 2-(3,5-dimethoxyphenyl)-N-[3-(4H-spiro[1,3-benzodioxine-2,4′-piperidin]-6-yl)phenyl]acetamide

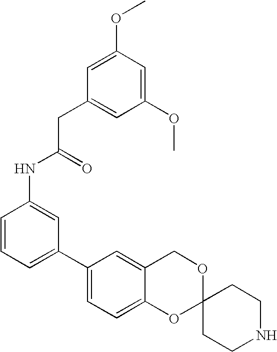

This compound was prepared using a similar procedure as for Example 12 to afford 34 mg of the product.

HPLC 100%, R_{T} 1.75 (System A 10-97% MeCN over 3 min), LC-MS ES⁺ m/z 475. ¹H NMR (500 MHz, CDCl₃) δ 1.74-1.91 (m, 4H), 2.85-2.97 (m, 4H), 3.61 (s, 2H), 3.73 (s, 6H), 4.82 (s, 2H), 6.36 (d, J=2.2 Hz, 1H), 6.41 (d, J=1.9 Hz, 2H), 6.84 (d, J=8.5 Hz, 1H), 7.09 (s, 1H), 7.15-7.21 (m, 2H), 7.27-7.32 (m, 1H), 7.58 (s, 1H).

### Example 29

### 2-[4-(dimethylamino)phenyl]-N-[3-(4H-spiro[1,3-benzodioxine-2,4′-piperidin]-6-yl)phenyl]acetamide

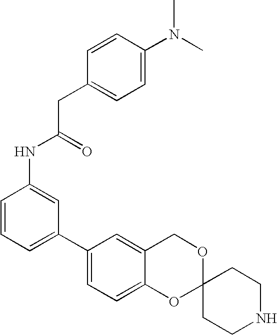

This compound was prepared using a similar procedure as for Example 12 to afford 12 mg of the product.

HPLC 100%, R_{T} 1.29 (System A 10-97% MeCN over 3 min), LC-MS ES⁺ m/z 230. ¹H NMR (500 MHz, CDCl₃) δ 1.83-1.89 (m, 4H), 2.93-3.03 (m, 10H), 3.65 (s, 2H), 4.89 (s, 2H), 6.75 (d, J=8.8 Hz, 2H), 6.90 (d, J=8.5 Hz, 1H), 7.09-7.24 (m, 5H), 7.29 (d, J=5 Hz, 2H), 7.36 (dd, J=8.3, 1.7 Hz, 1H), 7.62 (s, 1H).

### Example 30

### N-[3-(4H-spiro[1,3-benzodioxine-2,4′-piperidin]-6-yl)phenyl]-2-(2,3,4-trimethoxyphenyl)acetamide

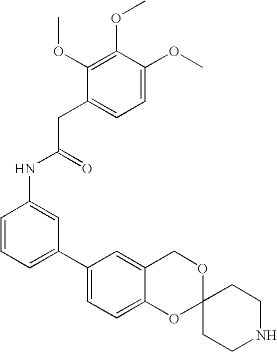

This compound was prepared using a similar procedure as for Example 12 to afford 20 mg of the product.

HPLC 100%, R_{T} 1.91 (System A 10-97% MeCN over 3 min), LC-MS ES⁺ m/z 505. ¹H NMR (400 MHz, CDCl₃) δ 1.79-1.97 (m, 4H), 2.90-3.02 (m, 4H), 3.63 (s, 2H), 3.85 (s, 3H), 3.88 (s, 3H), 3.97 (s, 3H), 4.88 (s, 2H), 6.66 (d, J=8.5 Hz, 1H), 6.89 (d, J=8.5 Hz, 1H), 6.98 (d, J=8.5 Hz, 1H), 7.15 (s, 1H), 7.20 (m, 1H), 7.26-7.30 (m, 2H), 7.32-7.38 (m, 1H), 7.70 (s, 1H), 7.83 (s, 1H).

### Example 31

### 2-(4-hydroxy-3,5-dimethoxyphenyl)-N-[3-(4H-spiro[1,3-benzodioxine-2,4′-piperidin]-6-yl)phenyl]acetamide

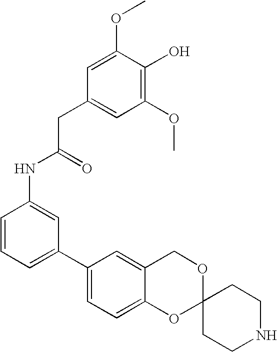

This compound was prepared using a similar procedure as for Example 12 to afford 14 mg of the product.

HPLC 100%, R_{T} 1.68 (System A 10-97% MeCN over 3 min), LC-MS ES⁺ m/z 491.

### Example 32

### 2-(4-hydroxy-3-methoxyphenyl)-N-[3-(4H-spiro[1,3-benzodioxine-2,4′-piperidin]-6-yl)phenyl]acetamide

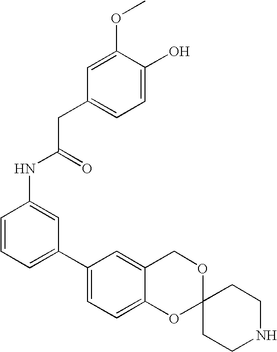

This compound was prepared using a similar procedure as for Example 12 to afford 6.5 mg of the product.

HPLC 100%, R_{T} 1.71 (System A 10-97% MeCN over 3 min), LC-MS ES⁺ m/z 461.

### Example 33

### N-[3-(4H-spiro[1,3-benzodioxine-2,4′-piperidin]-6-yl)phenyl]-2-[3-(trifluoromethyl)phenyl]acetamide

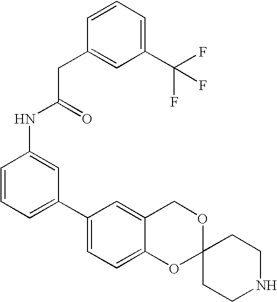

This compound was prepared using a similar procedure as for Example 12 to afford 20 mg of the product.

HPLC 100%, R_{T} 2.16 (System A 10-97% MeCN over 3 min), LC-MS ES⁺ m/z 483. ¹H NMR (400 MHz, MeOD) δ 1.81-1.95 (m, 4H), 2.85-2.98 (m, 4H), 3.79 (s, 2H), 4.90 (s, 2H), 6.87 (d, J=8.5 Hz, 1H), 7.22-7.36 (m, 3H), 7.36-7.47 (m, 2H), 7.48-7.59 (m, 2H), 7.59-7.65 (m, 1H), 7.55 (s, 1H), 7.80 (s, 1H).

### Example 34

### 2-(6-methoxy-1-benzofuran-3-yl)-N-[3-(4H-spiro[1,3-benzodioxine-2,4′-piperidin]-6-yl)phenyl]acetamide

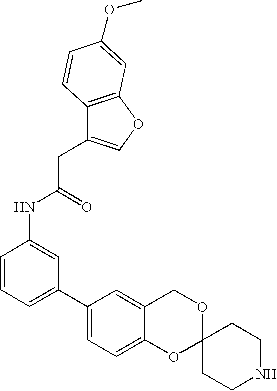

This compound was prepared using a similar procedure as for Example 12 to afford 16 mg of the product.

HPLC 100%, R_{T} 2.03 (System A 10-97% MeCN over 3 min), LC-MS ES⁺ m/z 485. ¹H NMR (500 MHz, MeOD) δ 1.84-1.95 (m, 4H), 2.87-2.99 (m, 4H), 3.75 (s, 2H), 3.81 (s, 3H), 4.90 (s, 2H), 6.87 (d, J=8.5 Hz, 1H), 7.04 (s, 1H), 7.24-7.36 (m, 3H), 7.38-7.46 (m, 2H), 7.51 (d, J=8.5 Hz, 1H), 7.62 (s, 1H), 7.80 (s, 1H).

### Example 35

### N-[3-(4H-spiro[1,3-benzodioxine-2,4′-piperidin]-6-yl)phenyl]-N′-(3,4,5-trimethoxyphenyl)urea

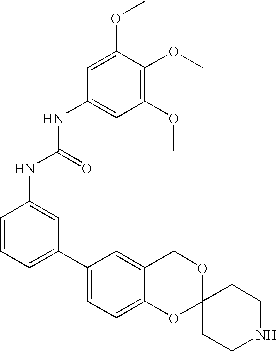

This compound was prepared using the general synthetic procedure G to afford 28 mg of the product.

HPLC 100%, R_{T} 1.16 (System A 30-80% MeCN over 3 min), LC-MS ES⁺ m/z 506. ¹H NMR (400 MHz, MeOD) δ 1.84-1.96 (m, 4H), 2.86-2.97 (m, 4H), 3.72 (s, 3H), 3.83 (s, 6H), 4.92 (s, 2H), 6.80 (s, 2H), 6.89 (d, J=8.5 Hz, 1H), 7.19-7.25 (m, 1H), 7.25-7.35 (m, 3H), 7.43 (dd, J=8.5, 2,3 Hz, 1H), 7.72 (t, J=1.8 Hz, 1H).

### Example 36

### N-[3-(4H-spiro[1,3-benzodioxine-2,4′-piperidin]-6-yl)phenyl]-N′-(3,4,5-trimethoxybenzyl)urea

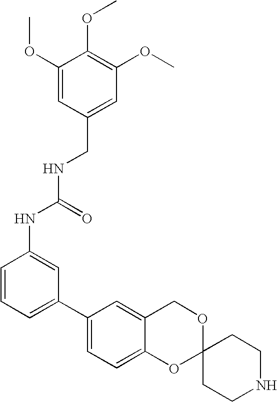

This compound was prepared using the general synthetic procedure G to afford 29 mg of the product.

HPLC 100%, R_{T} 1.10 (System A 30-80% MeCN over 3 min), LC-MS ES⁺ m/z 520. ¹H NMR (400 MHz, MeOD) δ 1.84-1.95 (m, 4H), 2.86-2.97 (m, 4H), 3.73 (s, 3H), 3.82 (s, 6H), 4.43 (s, 2H), 4.91 (s, 2H), 6.65 (s, 2H), 6.87 (d, J=8.3 Hz, 1H), 7.14-7.20 (m, 1H), 7.21-7.33 (m, 3H), 7.41 (dd, J=8.5, 2.3 Hz, 1H), 7.64 (t, J=1.8 Hz, 1H).

### Example 37

### AC3566003, N-(2-methylquinolin-6-yl)-N′-[3-(4H-spiro[1,3-benzodioxine-2,4′-piperidin]-6-yl)phenyl]urea

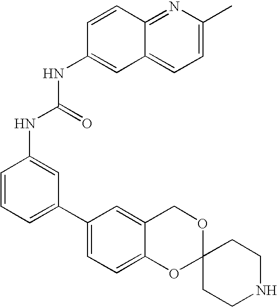

This compound was prepared using the general synthetic procedure G to afford 13 mg of the product.

HPLC 100%, R_{T} 1.54 (System A 10-97% MeCN over 3 min), HPLC 95%, R_{T} 1.37 (System B 10-97% MeCN over 3 min). ¹H NMR (400 MHz, MeOD) δ 1.90 (m, 4H), 2.67 (3, 3H), 2.92 (m, 4H), 4.92 (s, 2H), 6.89 (d, J=8.3 Hz, 1H), 7.24 (m, 1H), 7.28-7.40 (m, 4H), 7.40-7.48 (m, 1H), 7.67 (dd, J=9.2, 2.4 Hz, 1H), 7.74 (s, 1H), 7.87 (d, J=9.0 Hz, 1H), 8.11 (m, 2H).

### Example 38

### N-[2-(5-methoxy-1H-indol-3-yl)ethyl]-3-(4H-spiro[1,3-benzodioxine-2,4′-piperidin]-6-yl)benzamide

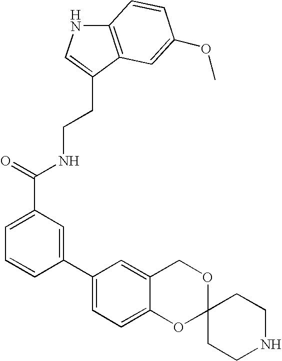

This compound was prepared using a similar procedure as for Example 12 to afford 4 mg of the product.

HPLC 100%, R_{T} 1.30 (System A 30-80% MeCN over 3 min), R_{T} 1.30 (System B 30-80% MeCN over 3 min), LC-MS ES⁺ m/z 250, 498. ¹H NMR (400 MHz, MeOD) δ 1.90 (m, 4H), 2.92 (m, 4H), 3.05 (m, 2H), 3.66 (m, 2H), 3.68 (s, 3H), 4.92 (s, 2H), 6.72 (dd, J=8.8, 2.3 Hz, 1H), 6.90 (d, J=8.5 Hz, 1H), 7.04-7.10 (m, 2H), 7.22 (d, J=8.8 Hz, 1H), 7.28 (d, J=3 Hz, 1H), 7.40-7.50 (m, 3H), 7.66-7.73 (m, 2H), 7.88 (d, J=1.6 Hz, 1H).

### Example 39

### 2-(5-methoxy-1H-indol-3-yl)-N-[2-methoxy-5-(4H-spiro[1,3-benzodioxine-2,4′-piperidin]-6-yl)phenyl]acetamide

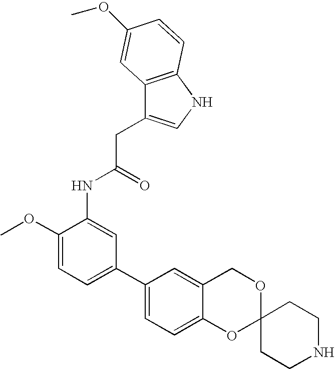

This compound was prepared using a similar procedure as for Example 12 to afford 10 mg of the product.

HPLC 100%, R_{T} 1.95 (System A 10-97% MeCN over 3 min), LC-MS ES⁺ m/z 514. ¹H NMR (400 MHz, CDCl₃) δ 1.73-1.99 (m, 7H), 2.98 (m, 4H), 3.54 (s, 2H), 3.82 (s, 3H), 3.89 (s, 2H), 4.87 (s, 2H), 6.76 (d, J=8.5 Hz, 1H), 6.89 (m, 2H), 7.05 (d, J=2.3 Hz, 1H), 7.13 (dd, J=8.5, 2.3 Hz, 1H), 7.16 (d, J=2.0 Hz, 1H), 7.20 (d, J=1.5 Hz, 1H), 7.31 (d, J=8.8 Hz, 1H), 7.36 (dd, J=8.5, 2.3 Hz, 1H), 8.14 (s, 1H), 8.24 (s, 1H), 8.58 (d, J=2.3 Hz, 1H).

### Example 40

### N-[2-methoxy-5-(4H-spiro[1,3-benzodioxine-2,4′-piperidin]-6-yl)phenyl]-2-(3,4,5-trimethoxyphenyl)acetamide

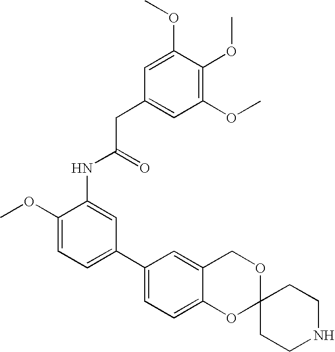

This compound was prepared using a similar procedure as for Example 12 to afford 18 mg of the product.

HPLC 95%, R_{T} 1.92 (System A 10-97% MeCN over 3 min), LC-MS ES⁺ m/z 535. ¹H NMR (400 MHz, CDCl₃) δ 1.69-2.01 (m, 7H), 2.97 (m, 4H), 3.70 (s, 2H), 3.77 (s, 3H), 3.85 (s, 3H), 3.87 (s, 6H), 4.88 (s, 2H), 6.55 (s, 2H), 6.84 (d, J=8.5 Hz, 1H), 6.88 (d, J=8.5 Hz, 1H), 7.12-7.20 (m, 2H), 7.36 (dd, J=8.5, 2.3 Hz, 1H), 7.93 (s, 1H), 8.58 (d, J=2.3 Hz, 1H).

### Example 41

### 3-(3,4-dimethoxyphenyl)-N-[2-methoxy-5-(4H-spiro[1,3-benzodioxine-2,4′-piperidin]-6-yl)phenyl]propanamide

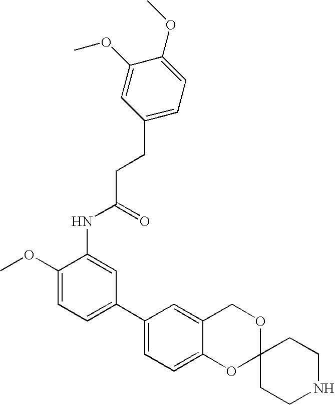

This compound was prepared using a similar procedure as for Example 12 to afford 15 mg of the product.

HPLC 96%, R_{T} 1.94 (System A 10-97% MeCN over 3 min), LC-MS ES⁺ m/z 519. ¹H NMR (400 MHz, CDCl₃) δ 1.82-2.01 (m, 5H), 2.70 (t, J=7.5 Hz, 2H), 2.91-3.07 (m, 6H), 3.82 (s, 3H), 3.84 (s, 3H), 3.84 (s, 3H), 4.89 (s, 2H), 6.70-6.82 (m, 3H), 6.88 (m, 2H), 7.13-7.22 (m, 2H), 7.39 (dd, J=8.5, 2.0 Hz, 1H), 7.68 (s, 1H), 8.63 (d, J=2.0 Hz, 1H).

### Example 42

### 2-(5-hydroxy-1H-indol-3-yl)-N-[3-(4H-spiro[1,3-benzodioxine-2,4′-piperidin]-6-yl)phenyl]acetamide

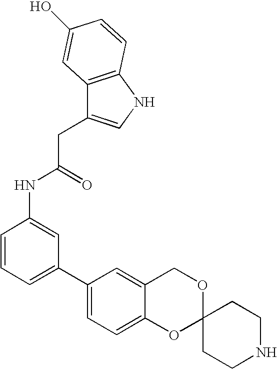

This compound was prepared using a similar procedure as for Example 12 to afford 22 mg of the product.

HPLC 100%, R_{T} 1.63 (System A 10-97% MeCN over 3 min), LC-MS ES⁺ m/z 470. ¹H NMR (400 MHz, CDCl₃-MeOD) δ 1.81-2.00 (m, 4H), 2.97 (m, 4H), 3.76 (s, 2H), 4.82 (s, 2H), 6.77 (dd, J=8.8, 2.2 Hz, 1H), 6.83 (d, J=8.6 Hz, 1H), 6.92 (d, J=2.3 Hz, 1H), 7.09 (d, J=2.3 Hz, 1H), 7.12-7.18 (m, 2H), 7.19-7.24 (m, 3H), 7.29 (dd, J=8.3, 2 Hz, 1H), 7.50 (s, 1H).

### Example 43

### N-[2-(1H-indol-3-yl)ethyl]-3-(4H-spiro[1,3-benzodioxine-2,4′-piperidin]-6-yl)benzamide

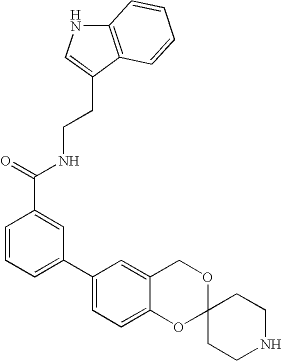

3-[1′-(tert-butoxycarbonyl)-4H-spiro[1,3-benzodioxine-2,4′-piperidin]-6-yl]benzoic acid (INTERMEDIATE 16), (34 mg, 0.08 mmol) was mixed with tryptamine (15 mg, 0.096 mmol), 6-methoxytryptamine (18 mg, 0.096 mmol) and 7-benzyloxytryptamine (26 mg, 0.096 mmol) and Et₃N (20 mg, 0.2 mmol) in DMF (0.8 mmol). Then TBTU (31 mg, 0.096 mmol) was added and the solution was left at r.t. overnight. Evaporated in a Genevac.

The crude amides were dissolved in DCM (0.8 mL) and TFA (0.2 mL) was added. The solution was evaporated in vacuum after ca 30 min. The residue was dissolved in MeOH and made alkaline with 25% aq. NH₃. Prep. HPLC on Xterra column with a gradient of 28-58, 27-57, 40-70% CH₃CN-0.05 M NH₄HCO₃, pH 10 over 6 min. Isolated 18 mg.

HPLC 100%, R_{T} 1.90 (System A 10-97% MeCN over 3 min), LC-MS ES⁺ m/z 468. ¹H NMR (400 MHz, CDCl₃) δ 1.83-2.01 (m), 3.03 (t, J=5.7 Hz, 4H), 3.11 (t, J=6.7 Hz, 2H), 3.82 (q, J=6.4 Hz, 2H), 4.89 (s, 2H), 6.24 (m, 1H), 6.93 (m, J=8.5 Hz, 1H), 7.06-7.18 (m, 3H), 7.22 (m, 1H), 7.32-7.44 (m, 3H), 7.55 (m, 1H), 7.60 (dd, J=7.7, 1.9 Hz, 1H), 7.66 (d, J=8.0 Hz, 1H), 7.81 (t, J=1.6 Hz, 1H), 8.09 (s, 1H).

### Example 44

### N-[2-(6-methoxy-1H-indol-3-yl)ethyl]-3-(4H-spiro[1,3-benzodioxine-2,4′-piperidin]-6-yl)berzamide

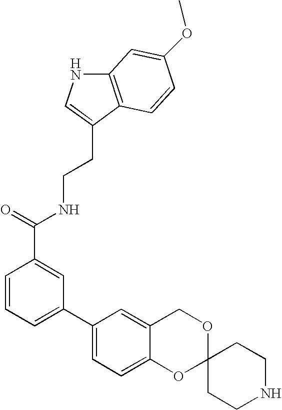

This compound was prepared using a similar procedure as for Example 43 to afford 4 mg of the product.

HPLC 100%, R_{T} 1.86 (System A 10-97% MeCN over 3 min), LC-MS ES⁺ m/z 498. ¹H NMR (400 MHz, CDCl₃) δ 1.80-2.13 (m), 2.98-3.11 (m, 6H), 3.79 (q, J=6.5 Hz, 2H), 3.83 (s, 3H), 4.90 (s, 2H), 6.24 (m, 1H), 6.78 (dd, J=8.8, 2.3 Hz, 1H), 6.87 (d, J=2.0 Hz, 1H), 6.93 (d, J=8.5 Hz, 1H), 6.96 (d, J=2.0 Hz, 1H), 7.16 (d, J=2.3 Hz, 1H), 7.32-7.44 (m, 2H), 7.51 (d, J=8.8 Hz, 1H), 7.53-7.57 (m, 1H), 7.58-7.63 (m, 1H), 7.81 (t, J=1.8 Hz, 1H), 7.94 (s, 1H).

### Example 45

### N-[2-(5-chloro-1H-indol-3-yl)ethyl]-3-(4H-spiro[1,3-benzodioxine-2,4′-piperidin]-6-yl)benzamide

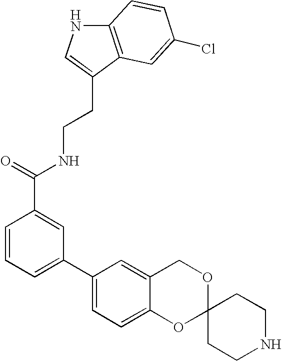

This compound was prepared using a similar procedure as for Example 43 to afford 25 mg of the product.

HPLC 100%, R_{T} 2.04 (System A 10-97% MeCN over 3 min), LC-MS ES⁺ m/z 502. ¹H NMR (400 MHz, CDCl₃) δ 1.81-1.99 (m, 4H), 2.96 (t, J=5.8 Hz, 4H), 3.01 (m, J=6.8 Hz, 2H), 3.70 (m, 2H), 4.85 (s, 2H), 6.68 (m, 1H), 6.88 (d, J=8.5 Hz, 1H), 7.04-7.09 (m, 2H), 7.13 (d, J=2 Hz, 1H), 7.24 (obscured by CHCl3), 7.33 (dd, J=8.4, 2.4 Hz, 1H), 7.37 (t, J=7.8 Hz, 1H), 7.50-7.59 (m, 3H), 7.77 (t, J=1.8 Hz, 1H).

### Example 46

### N-[2-(5-fluoro-1H-indol-3-yl)ethyl]-3-(4H-spiro[1,3-benzodioxine-2,4′-piperidin]-6-yl)benzamide

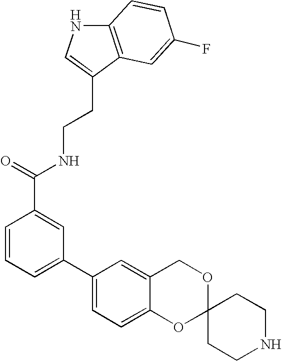

This compound was prepared using a similar procedure as for Example 43 to afford 25 mg of the product.

HPLC 100%, R_{T} 1.95 (System A 10-97% MeCN over 3 min), LC-MS ES⁺ m/z 486. ¹H NMR (400 MHz, CDCl₃) δ 1.82-1.99 (m, 4H), 2.92-3.03 (m, 4H), 3.69 (m, 2H), 4.85 (s, 2H), 6.76 (m, 1H), 6.83-6.90 (m, 2H), 7.07 (s, 1H), 7.13 (d, J=2.3 Hz, 1H), 7.18-7.24 (m, 2H)), 7.33 (dd, J=8.5, 2.3 Hz, 1H), 7.36 (t, J=7.8 Hz, 1H), 7.50-7.59 (m, 2H), 7.76 (t, J=1.8 Hz, 1H).

### Example 47

### N-[2-(5-methoxy-1H-indol-3-yl)ethyl]-5-(4H-spiro[1,3-benzodioxine-2,4′-piperidin]-6-yl)nicotinamide

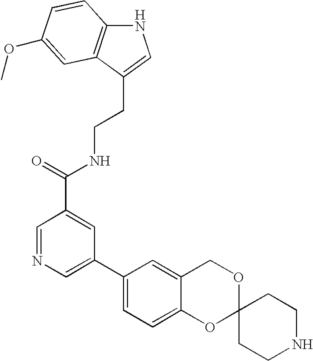

5-bromo-N-[2-(5-methoxy-1H-indol-3-yl)ethyl]nicotinamide (INTERMEDIATE 17) (51 mg, 0.13 mmol), bis(neopentyl glycolato)diboron (68 mg, 0.30 mmol), KOAc (88 mg, 0.9 mmol) and PdCl2dppf (10 mg, 0.014 mmol) were mixed in DME (2 mL) and heated to 100° C. in a capped vial for 1 h. tert-Butyl 6-bromo-1′H,4H-spiro[1,3-benzodioxine-2,4′-piperidine]-1′-carboxylate (INTERMEDIATE 13) (77 mg, 0.2 mmol), NaHCO₃ (34 mg, 0.4 mmol), Pd(PPh₃)₄ (7 mg, 0.006 mmol) and water (1 mL) and DME (1 mL) was added and the mixture was heated to 100° C. for 80 min. The reaction mixture was diluted with EtOH and filtered on Celite. The solvent was evaporated and the crude product was purifed by flash-chromatography on silica gel with 2% MeOH/CHCl₃. This product was treated with TFA (0.25 mL) and DCM (1 mL) over 30 min. The solution was evaporated in vacuum and the residue was dissolved in MeOH and made alkaline with 25% aq. NH₃. Prep. HPLC on Xterra column with a gradient of 21-51% CH₃CN-0.05 M NH₄HCO₃, pH 10 over 6 min. This gave 22 mg of the title product.

HPLC 100%, R_{T} 1.57 (System A 10-97% MeCN over 3 min), LC-MS ES⁺ m/z 499. ¹H NMR (400 MHz, MeOD) δ 1.94 (m, 4H), 2.97 (m, 4H), 3.06 (t, J=7.1 Hz, 2H), 3.69 (m, 5H), 4.94 (s, 2H), 6.72 (dd, J=8.8, 2.5 Hz, 1H), 6.96 (d, J=8.5 Hz, 1H), 7.05 (d, J=2.3 Hz, 1H), 7.09 (s, 1H), 7.22 (d, J=8.8 Hz, 1H), 7.35 (d, J=2.3 Hz, 1H), 7.48 (dd, J=8.5, 2.3 Hz, 1H), 8.22 (t, J=2.1 Hz, 1H), 8.80 (d, J=2.0 Hz, 1H), 8.85 (d, J=2.1 Hz, 1H).

### Example 48

### N-[5-(4H-spiro[1,3-benzodioxine-2,4′-piperidin]-6-yl)pyridin-3-yl]-2-(3,4,5-trimethoxyphenyl)acetamide

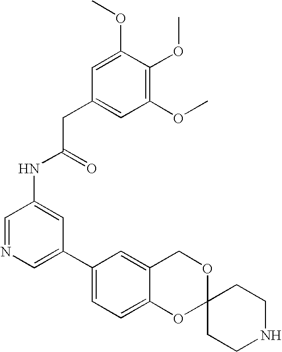

This compound was prepared using a similar procedure as for Example 47 to afford 6 mg of the product.

HPLC 90%, R_{T} 1.44 (System A 10-97% MeCN over 3 min), LC-MS ES⁺ m/z 506. ¹H NMR (400 MHz, MeOD) δ 1.92 (m, 4H), 2.95 (m, 4H), 3.67 (s, 2H), 3.74 (s, 3H), 3.84 (s, 6H), 4.92 (s, 2H), 6.88 (s, 2H), 6.94 (d, J=8.5 Hz, 1H), 7.33 (d, J=2.0 Hz, 1H), 7.75 (dd, J=8.5, 2.3 Hz, 1H), 8.32 (t, J=2.1 Hz, 1H), 8.46 (d, J=2.0 Hz, 1H), 8.62 (d, J=2.3 Hz, 1H).

### Example 49

### 2-(5-methoxy-1H-indol-3-yl)-N-[5-(4H-spiro[1,3-benzodioxine-2,4′-piperidin]-6-yl)pyridin-3-yl]acetamide

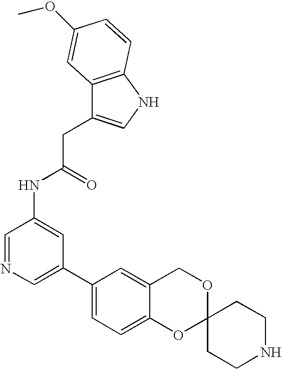

This compound was prepared using a similar procedure as for Example 47 to afford 25 mg of the product.

HPLC 98%, R_{T} 1.45 (System A 10-97% MeCN over 3 min), LC-MS ES⁺ m/z 485. ¹H NMR (400 MHz, MeOD) δ 1.91 (m, 4H), 2.95 (m, 4H), 3.79 (s, 3H), 3.83 (s, 2H), 4.90 (s, 2H), 6.77 (dd, J=8.8, 2.3 Hz, 1H), 6.92 (d, J=8.5 Hz, 1H), 7.12 (d, J=2.5 Hz, 1H), 7.21 (s, 1H), 7.24 (d, J=8.8 Hz, 1H), 7.31 (d, J=2.0 Hz, 1H), 7.43 (dd, J=8.5, 2.3 Hz, 1H), 8.30 (t, J=2.1 Hz, 1H), 8.44 (d, J=2.0 Hz, 1H), 8.60 (d, J=2.3 Hz, 1H).

### Example 50

### N-[2-(5-methoxy-1H-indol-3-yl)ethyl]-4-(4H-spiro[1,3-benzodioxine-2,4′-piperidin]-6-yl)pyridine-2-carboxamide

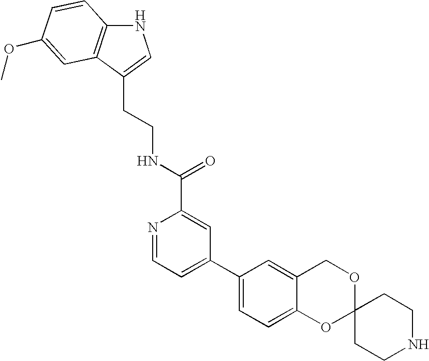

This compound was prepared using a similar procedure as for Example 43 to afford 3 mg of the product.

HPLC 100%, R_{T} 1.84 (System A 10-97% MeCN over 3 min), LC-MS ES⁺ m/z 499. ¹H NMR (400 MHz, CDCl₃) δ 1.89-1.96 (m, 4H), 2.91-2.98 (m, 4H), 3.05 (t, J=7.2 Hz, 2H), 3.73 (t, J=7.2 Hz, 2H), 4.95 (s, 2H), 6.73 (dd, J=8.8, 2.3 Hz, 1H), 6.97 (d, J=8.5 Hz, 1H), 7.07-7.12 (m, 2H), 7.21 (d, J=8.8 Hz, 1H), 7.51 (d, J=2.5 Hz, 1 H), 7.62 (dd, J=8.5, 2.5 Hz, 1H), 7.73 (dd, J=5.3, 2.0 Hz, 1H), 8.28 (d, J=1.3 Hz, 1H), 8.65 (d, J=4.5 Hz, 1H).

### Example 51

### 2-fluoro-5-(4H-spiro[1,3-benzodioxine-2,4′-piperidin]-6-yl)-N-(3,4,5-trimethoxybenzyl)benzamide

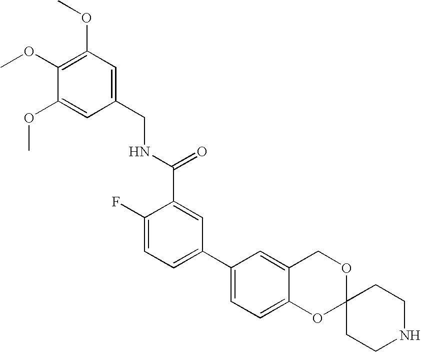

This compound was prepared using a similar procedure as for Example 43 to afford 17 mg of the product.

HPLC 100%, R_{T} 1.87 (System A 10-97% MeCN over 3 min), LC-MS ES⁺ m/z 523.

### Example 52

### 2-fluoro-N-[2-(5-methoxy-1H-indol-3-yl)ethyl]-5-(4H-spiro[1,3-benzodioxine-2,4′-piperidin]-6-yl)benzamide

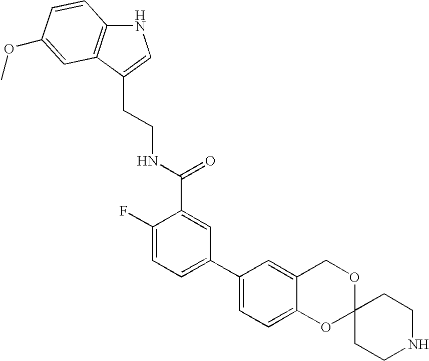

This compound was prepared using a similar procedure as for Example 43, but from Intermediate 23, to afford 8 mg of the product.

HPLC 100%, R_{T} 1.94 (System A 10-97% MeCN over 3 min), LC-MS ES⁺ m/z 516.

### Example 53

### 2-(1H-benzimidazol-1-yl)-N-[3-(4H-spiro[1,3-benzodioxine-2,4′-piperidin]-6-yl)phenyl]acetamide

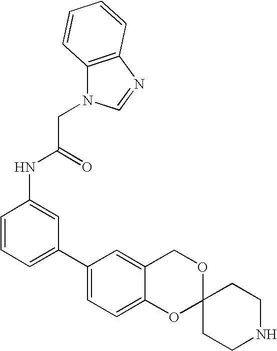

This compound was prepared using a similar procedure as for Example 12 to afford 17 mg of the product.

HPLC 100%, R_{T} 1.49 (System A 10-97% MeCN over 3 min), LC-MS ES⁺ m/z 455.

### Example 54

### 2-(5-methyl-2-phenyl-1,3-thiazol-4-yl)-N-[3-(4H-spiro[1,3-benzodioxine-2,4′-piperidin]-6-yl)phenyl]acetamide

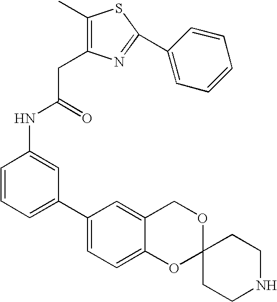

This compound was prepared using a similar procedure as for Example 12 to afford 19 mg of the product.

HPLC 100%, R_{T} 2.19 (System A 10-97% MeCN over 3 min), LC-MS ES⁺ m/z 512.

### Example 55

### 2-(4-tert-butylphenyl)-N-[3-(4H-spiro[1,3-benzodioxine-2,4′-piperidin]-6-yl)phenyl]acetamide

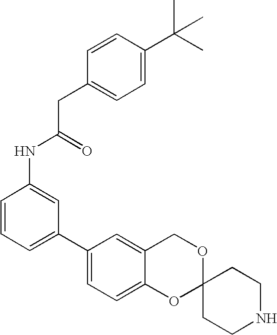

This compound was prepared using a similar procedure as for Example 12 to afford 23 mg of the product.

HPLC 100%, R_{T} 2.32 (System A 10-97% MeCN over 3 min), LC-MS ES⁺ m/z 471.

### Example 56

### 2-(5-ethyl-1H-indol-3-yl)-N-[3-(4H-spiro[1,3-benzodioxine-2,4′-piperidin]-6-yl)phenyl]acetamide

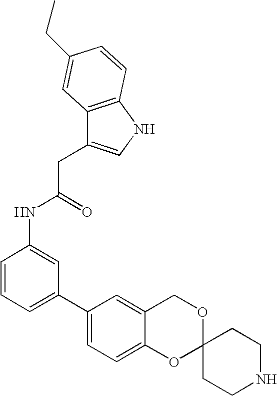

This compound was prepared using a similar procedure as for Example 12 to afford 12 mg of the product.

HPLC 97%, R_{T} 2.08 (System A 10-97% MeCN over 3 min), LC-MS ES⁺ m/z 482. ¹H NMR (400 MHz, MeOD) δ 1.24 (t, 3H), 1.90 (m, 4H), 2.70 (q, 2H), 2.93 (t, 4H), 3.81 (s, 2H), 4.90 (s, 2H), 6.87 (d, 1H), 6.97 (dd, 1H), 7.20 (s, 1H), 7.23-7.35 (m, 4H), 7.36-7.46 (m, 3H), 7.77 (m, 1H).

### Example 57

### 3-(1H-benzimidazol-1-yl)-N-[3-(4H-spiro[1,3-benzodioxine-2,4′-piperidin]-6-yl)phenyl]propanamide

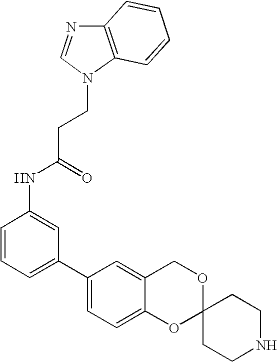

This compound was prepared using a similar procedure as for Example 12 to afford 19 mg of the product.

HPLC 100%, R_{T} 1.49 (System A 10-97% MeCN over 3 min), LC-MS ES⁺ m/z 469.

### Example 58

### 2-(2H-1,2,3-benzotriazol-2-yl)-N-[3-(4H-spiro[1,3-benzodioxine-2,4′-piperidin]-6-yl)phenyl]acetamide

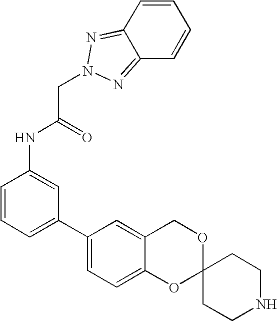

This compound was prepared using a similar procedure as for Example 12 to afford 17 mg of the product.

HPLC 100%, R_{T}1.91 (System A 10-97% MeCN over 3 min), LC-MS ES⁺ m/z 456.

### Example 59

### 3-(2-phenyl-1H-imidazol-1-yl)-N-[3-(4H-spiro[1,3-benzodioxine-2,4′-piperidin]-6-yl)phenyl]propanamide

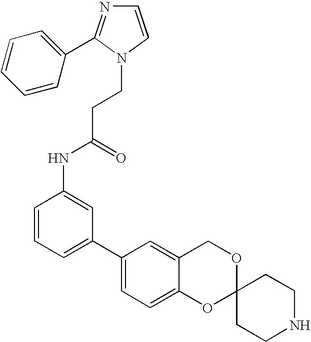

This compound was prepared using a similar procedure as for Example 12 to afford 12 mg of the product.

HPLC 100%, R_{T} 1.52 (System A 10-97% MeCN over 3 min), LC-MS ES⁺ m/z 495.

### Example 60

### 3-(2,3-dihydro-1-benzofuran-5-yl)-N-[3-(4H-spiro[1,3-benzodioxine-2,4′-piperidin]-6-yl)phenyl]propanamide

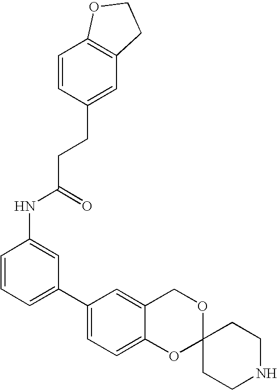

This compound was prepared using a similar procedure as for Example 12 to afford 20 mg of the product.

HPLC 100%, R_{T} 1.97 (System A 10-97% MeCN over 3 min), LC-MS ES⁺ m/z 471.

### Example 61

### 2-(3-pyridin-3-ylphenyl)-N-[3-(4H-spiro[1,3-benzodioxine-2,4′-piperidin]-6-yl)phenyl]acetamide

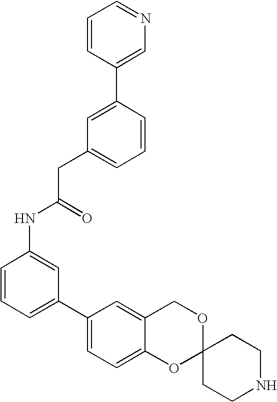

This compound was prepared using a similar procedure as for Example 12 to afford 20 mg of the product.

HPLC 99%, R_{T} 1.56 (System A 10-97% MeCN over 3 min), LC-MS ES⁺ m/z 492.

### Example 62

### N-[3-(1′-isopropyl-4H-spiro[1,3-benzodioxine-2,4′-piperidin]-6-yl)phenyl]-2-(3,4,5-trimethoxyphenyl)acetamide

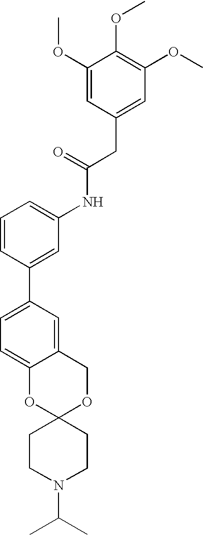

tert-butyl 6-(3-{[(3,4,5-trimethoxyphenyl)acetyl]amino}phenyl)-1′H,4H-spiro[1,3-benzodioxine-2,4′-piperidine]-1′-carboxylate (50 mg, 0.083 mmol) was dissolved in dichloromethane (3 mL) and TFA (0.5 mL) was added. The mixture was stirred for 30 minutes and evaporated. The crude product was dissolved in dichloromethane (2 mL) and 5 drops of triethylamine was added and the mixture was evaporated. The crude product was dissolved in acetone (3 mL) and NaCNBH₃ (20 mg, 0.32 mmol) was added. The mixture was stirred overnight and evaporated. The crude product was purified by flash chromatography using 2.5% MeOH in dichloromethane with a few drops of NEt₃ as the eluent. This gave 24 mg (53%) of a white solid.

HPLC 97% Rt=1.9 min (system A. 10-97% MeCN over 3 minutes). HPLC 97% Rt=1.7 min (system B. 10-97% MeCN over 3 minutes). MS (electronspray; [M+H]+) m/z 547.4. ¹H NMR (400 MHz, CHLOROFORM-D δ ppm 1.11 (d, J=6.78 Hz, 6H) 2.02 (d, J=8.03 Hz, 4H) 2.69 (t, J=5.27 Hz, 4H) 2.83-2.92 (m, 1H) 3.69 (s, 2H) 3.84-3.88 (m, 9H) 4.88 (s, 2H) 6.54 (s, 2H) 6.91 (d, J=8.53 Hz, 1H) 7.16-7.20 (m, J=1.76 Hz, 2H) 7.29-7.40 (m, 4H) 7.64 (s, 1H).

### Example 63

### 3-(3-chloro-4-methoxyphenyl)-N-[3-(4H-spiro[1,3-benzodioxine-2,4′-piperidin]-6-yl)phenyl]propanamide

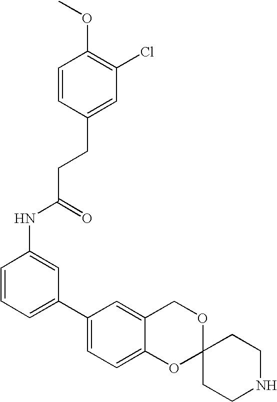

This compound was prepared using a similar procedure as for Example 12 to afford 18 mg of the product.

HPLC 100%, R_{T} 2.08 (System A 10-97% MeCN over 3 min), LC-MS ES⁺ m/z 493.

### Example 64

### 2-(1H-benzimidazol-2-yl)-N-[3-(4H-spiro[1,3-benzodioxine-2,4′-piperidin]-6-yl)phenyl]acetarmide

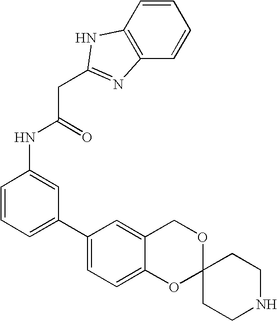

This compound was prepared using a similar procedure as for Example 12 to afford 8 mg of the product.

HPLC 98%, R_{T} 1.47 (System A 10-97% MeCN over 3 min), LC-MS ES⁺ m/z 455.

### Example 65

### 3-(1H-1,2,3-benzotriazol-1-yl)-N-[3-(4H-spiro[1,3-benzodioxine-2,4′-piperidin]-6-yl)phenyl]propanamide

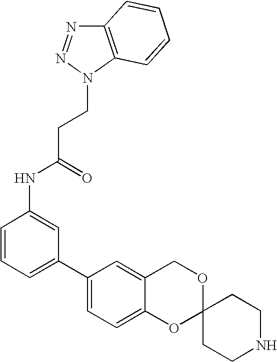

This compound was prepared using a similar procedure as for Example 12 to afford 17 mg of the product.

HPLC 100%, R_{T} 1.76 (System A 10-97% MeCN over 3 min), LC-MS ES⁺ m/z 470.

### Example 66

### 3-(1H-indazol-1-yl)-N-[3-(4H-spiro[1,3-benzodioxine-2,4′-piperidin]-6-yl)phenyl]propanamide

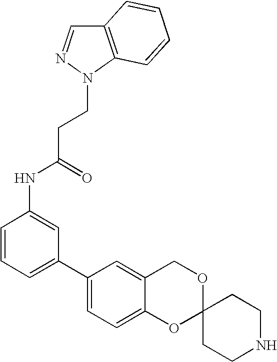

This compound was prepared using a similar procedure as for Example 12 to afford 18 mg of the product.

HPLC 100%, R_{T} 1.89 (System A 10-97% MeCN over 3 min), LC-MS ES⁺ m/z 469.

### Example 67

### 2-(2-methyl-1H-benzimidazol-1-yl)-N-[3-(4H-spiro[1,3-benzodioxine-2,4′-piperidin]-6-yl)phenyl]acetamide

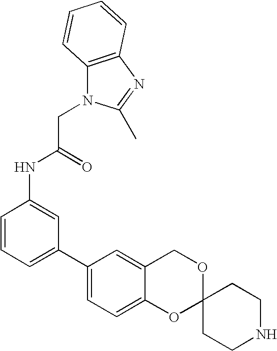

This compound was prepared using a similar procedure as for Example 12 to afford 19 mg of the product.

HPLC 100%, R_{T} 1.65 (System A 10-97% MeCN over 3 min), LC-MS ES⁺ m/z 469.

### Example 68

### 3-(2-methyl-1H-benzimidazol-1-yl)-N-[3-(4H-spiro[1,3-benzodioxine-2,4′-piperidin]-6-yl)phenyl]propanamide

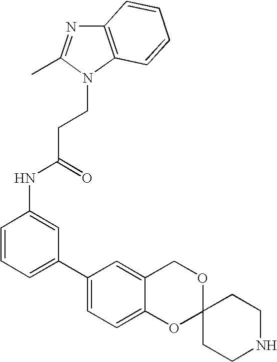

This compound was prepared using a similar procedure as for Example 12 to afford 19 mg of the product.

HPLC 100%, R_{T} 1.63 (System A 10-97% MeCN over 3 min), LC-MS ES⁺ m/z 483.

### Example 69

### 2-pyridin-2-yl-N-[3-(4H-spiro[1,3-benzodioxine-2,4′-piperidin]-6-yl)phenyl]acetamide

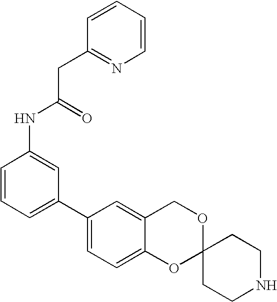

This compound was prepared using a similar procedure as for Example 12 to afford 4 mg of the product.

HPLC 100%, R_{T} 2.07 (System A 10-97% MeCN over 3 min), LC-MS ES⁺ m/z 415. ¹H NMR (500 MHz, DMSO-d6) δ 1.77 (m, 4H), 2.78 (m, 4H), 4.89 (s, 2H), 6.91 (d, J=8.5 Hz, 1H), 7.28 (m, 3H), 7.35 (t, J=7.9 Hz, 1H), 7.40 (d, J=7.9 Hz, 2H), 7.50 (d, J=7.9 Hz, 1H), 7.76 (t, J=7.6 Hz, 1H), 7.90 (s, 1H), 8.50 (d, J=4 Hz, 1H), 10.27 (s, 1H).

### Example 70

### 2-pyridin-4-yl-N-[3-(4H-spiro[1,3-benzodioxine-2,4′-piperidin]-6-yl)phenyl]acetamide,

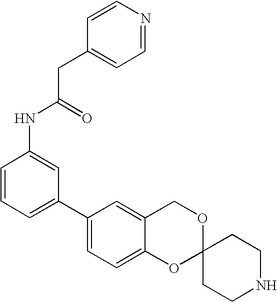

This compound was prepared using a similar procedure as for Example 12 to afford 12 mg of the product.

HPLC 100%, R_{T} 2.02 (System A 10-97% MeCN over 3 min), LC-MS ES⁺ m/z 415. ¹H NMR (500 MHz, DMSO-d6) δ 1.75 (m, 4H), 2.76 (m, 4H), 3.72 (s, 2H), 4.88 (s, 2H), 6.91 (d, J=8.5 Hz, 1H), 7.28 (m, 2H), 7.37 (m, 4H), 7.48 (d, J=7.9 Hz, 2H), 7.86 (s, 1H), 8.51 (d, J=5.5 Hz, 2H), 10.28 (s, 1H).

### Example 71

### 3-(1H-benzimidazol-2-yl)-N-[3-(4H-spiro[1,3-benzodioxine-2,4′-piperidin]-6-yl)phenyl]propanamide

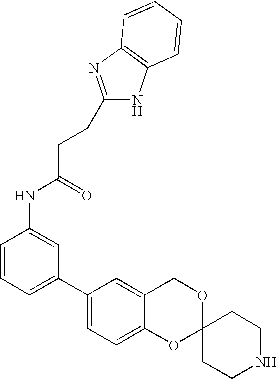

This compound was prepared using a similar procedure as for Example 12 to afford 15 mg of the product.

HPLC 100%, R_{T} 2.13 (System A 10-97% MeCN over 3 min), LC-MS ES⁺ m/z 468.

### Example 72

### N-methyl-N-(2-oxo-2-{[3-(4H-spiro[1,3-benzodioxine-2,4′-piperidin]-6-yl)phenyl]amino}ethyl)benzamide

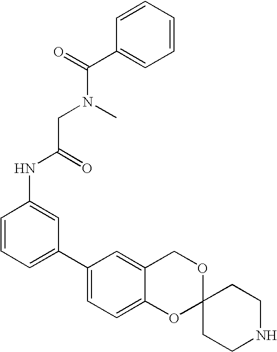

This compound was prepared using a similar procedure as for Example 12 to afford 17 mg of the product.

HPLC 100%, R_{T} 2.17 (System A 10-97% MeCN over 3 min), LC-MS ES⁺ m/z 471. ¹H NMR (500 MHz, DMSO-d6) δ 1.76 (m, 4H), 2.77 (m, 4H), 3.00 (m, 3H), 4.06 (s, 1H), 4.29 (s, 1H), 4.90 (s, 2H), 6.91 (d, J=8.5 Hz, 1H), 7.23-7.53 (m, 10H), 7.79-7.98 (m, 1H), 9.99 (s, 0.4H), 10.15 (s, 0.5H).

### Example 73

### N-[2-(3,4-dimethoxyphenyl)ethyl]-2-methyl-3-(4H-spiro[1,3-benzodioxine-2,4′-piperidin]-6-yl)benzamide

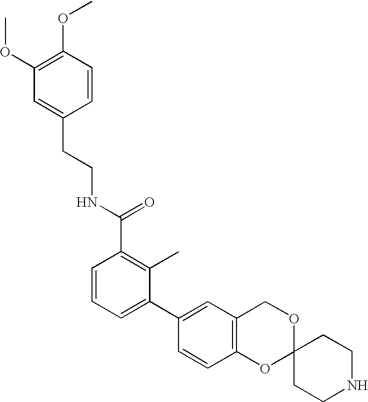

This compound was prepared using a similar procedure as for Example 12 to afford 25 mg of the product.

HPLC 100%, R_{T} 2.22 (System A 10-97% MeCN over 3 min), LC-MS ES⁺ m/z 502. ¹H NMR (500 MHz, DMSO-d6) δ 1.76 (m, 4H), 2.11 (s, 3H), 2.77 (m, 6H), 3.46 (m, 2H), 3.71 (s, 3H), 3.74 (s, 3H), 4.86 (s, 2H), 6.76 (d, J=8 Hz, 1H), 6.83-6.91 (m, 3H), 6.97 (s, 1H), 7.07 (d, J=5 Hz, 1H), 7.18 (m, 2H), 7.24 (m, 1H), 8.28 (t, J=5 Hz, 1H).

### Example 74

### 2-methyl-3-(4H-spiro[1,3-benzodioxine-2,4′-piperidin]-6-yl)-N-(3,4,5-trimethoxybenzyl)benzamide

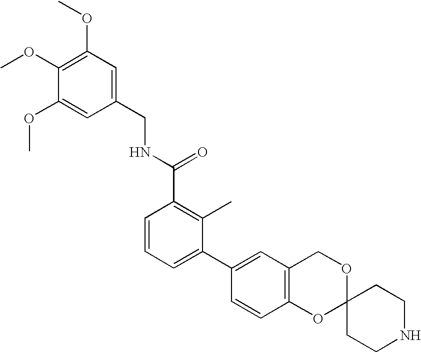

This compound was prepared using a similar procedure as for Example 12 to afford 23 mg of the product.

HPLC 100%, R_{T} 2.20 (System A 10-97% MeCN over 3 min), LC-MS ES⁺ m/z 518. ¹H NMR ((500 MHz, DMSO-d6) δ 1.76 (m, 4H), 2.20 (s, 3H), 2.77 (m, 4H), 3.64 (s, 3H), 3.76 (s, 6H), 4.39, 4.40 (s, 2H), 4.86 (s, 2H), 6.66 (s, 2H), 6.88 (d, J=8.5 Hz, 1H), 6.99 (s, 1H), 7.09 (d, J=5 Hz, 1H), 7.16-7.24 (m, 1H), 7.25-7.32 (m, 2H), 7.52-7.67 (m, 1H), 8.77 (t, J=5 Hz, 1H).

### Example 75

Kᵢ Determination

Human embryonic kidney (HEK293EBNA) cells stably expressing OX-1R seeded in 96- or 384-well plates are pre-loaded with Ca²⁺ sensing probe Fluo-4AM fluorescent dye for 60 min before addition of test compounds (10 μM for primary screen). Fluorescent intensity, which is a measurement of Ca²⁺ concentration inside the cells, is recorded using a Fluorometric imaging plate reader (FLIPR 98R 96-well format or FLIPR 3, 384-well format, Molecular Devices) and inhibition of the peak response evoked by orexin-A (EC₇₀ concentration) is calculated. Potency (Kᵢ) determinations are performed utilizing the same functional assay as described for primary screening, applying the compounds in the concentration range of 340 pM to 20 μM and recording the concentration resulting in a 50% inhibition of orexin-A induced Ca²⁺ release (IC₅₀) and from there calculating the inhibition constant (Kᵢ).

The calculation of the functional Kᵢ values for the inhibitors was performed by use of Activity Base. The Kᵢ value is calculated from IC₅₀ using the Cheng Prushoff equation (with reversible inhibition that follows the Michaelis-Menten equation): Kᵢ=IC₅₀/(1+[S]/Km) (Cheng, Y. C. & Prushoff, W. H. (1973) Biochem. Pharmacol. 22:3099-3108). Compounds of Formula (I) exhibit Kᵢ values for human OX-1R in the range from 30 nM to ≧2 μM (See Table I).

TABLE I

| Example No. | Kᵢ (nM) |
|---|---|
| 2 | 349 |
| 4 | 823 |
| 8 | 253 |

1. A compound of the Formula I, wherein 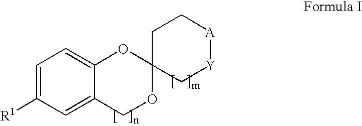
n and m are, independently, 0 or 1;
A and Y are independently CH₂, O or NR², wherein R² is H or C₁-C₆ alkyl, provided that one of A and Y is CH₂, and the other one is O or NR², and provided that when m is 0, then Y is CH₂; 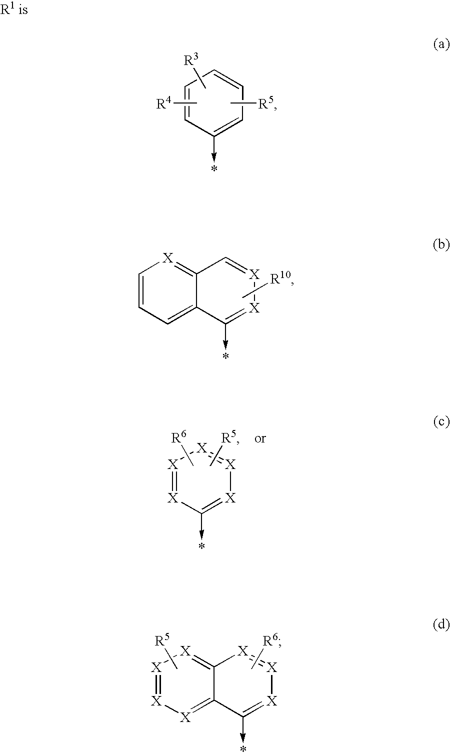
X is CH or N,
provided that when R¹ is (c) or (d), not more than two of the groups X are N;
R³ and R⁴ are independently C₁-C₆ alkoxy;
R⁵ is H, halogen, C₁-C₆ alkyl, or C₁-C₆ alkoxy;
R⁶, which is bonded to R¹ in a position wherein X is CH, is
(a) —R⁷—C(O)—(CH₂)ₚ—Ar,
(b) —R⁷—C(O)—CH₂—O—Ar,
(c) —R⁷—C(O)—CH₂—S—Ar,
(d) —R⁷—(CH₂)ₚ—C(O)—NH—R⁸,
(e) —R⁷—(CH₂)ₚ—C(O)—R⁹,
(f) —R⁷—(CH₂)ₚ—R⁹, or
(g) —C(O)—NH—(CH₂)ₚ—Ar,
wherein p is an integer 1 or 2;
R⁷ is O or NH;
R⁸ is H or Ar;
Ar is aryl or heteroaryl, Ar being optionally substituted with one or more of halogen, C₁-C₆ alkyl, C₁-C₆ alkoxy, di-(C₁-C₃)alkylamino, or when Ar is indole, MeSO₂; 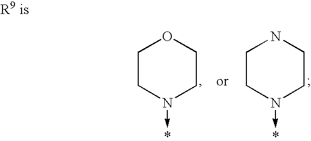
R¹⁰, which is bonded to R¹ in a position wherein X is CH, is H or NH₂;
and pharmaceutically acceptable salts, hydrates, solvates, geometrical isomers, tautomers, optical isomers, N-oxides and prodrug forms thereof.

2. The compound according to claim 1 wherein n is 1.

3. The compound according to claim 1 wherein m is 1.

4. The compound according to claim 1 wherein A is NH.

5. The compound according to claim 1 wherein Y is CH₂.

6. The compound according to claim 1 wherein R¹ is (c) and substituted with R⁶.

7. The compound according to claim 1 wherein R¹ is phenyl and substituted with R⁶.

8. The compound according to claim 1 wherein R⁶ is (a), (b) or (c) and R⁷ is NH.

9. The compound according to claim 1 wherein R⁶ is (d), (e) or (f) and R⁷ is 0.

10. The compound according to claim 1 wherein R⁶ is (a), (d), (e) or (f) and p is 1.

11. The compound according to claim 1 wherein R⁶ is (d) and R⁹ is Ar.

12. The compound according to claim 1 wherein Ar is phenyl or indole.

13. The compound according to claim 1 wherein Ar is substituted with one or more C₁-C₆ alkoxy groups.

14. A compound according to claim 1 having the Formula II: 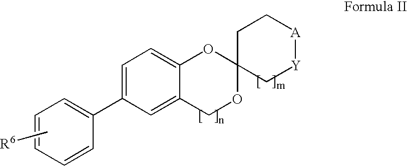
wherein A, Y, R⁶, n, and m are as defined in claim 1.

15. A compound according to claim 14 having the Formula III: 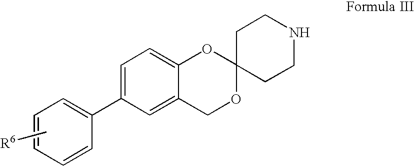
wherein R⁶ is as defined in claim 1.

16. The compound according to claim 1, which compound is selected from the group consisting of:
5-(3,4,5-trimethoxyphenyl)spiro[1,3-benzodioxole-2,4′-piperidine];
5-(2′,3′,5′,6′-tetrahydro-4H-spiro[1,3-benzodioxine-2,4′-pyran]-6-yl)quinoline;
6-(3,4,5-trimethoxyphenyl)-4H-spiro[1,3-benzodioxine-2,3′-piperidine];
N-(2,6-dimethylphenyl)-2-[3-(4H-spiro[1,3-benzodioxine-2,4′-piperidin]-6-yl)phenoxy]acetamide;
N-phenyl-2-[3-(4H-spiro[1,3-benzodioxine-2,4′-piperidin]-6-yl)phenoxy]acetamide;
2-(5-methoxy-1H-indol-3-yl)-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-2-(3,4,5-trimethoxyphenyl)acetamide;
2-(4-fluorophenoxy)-N-[3-(4H-spiro[1,3-benzodioxine-2,4′-piperidin]-6-yl)phenyl]acetamide;
1-(4H-spiro[1,3-benzodioxine-2,4′-piperidin]-6-yl)isoquinolin-3-amine;
2-[2-(4H-spiro[1,3-benzodioxine-2,4′-piperidin]-6-yl)phenoxy]acetamide;
2-[3-(4H-spiro[1,3-benzodioxine-2,4′-piperidin]-6-yl)phenoxy]-N-[2-(trifluoromethyl)phenyl]acetamide;
6-[3-(2-morpholin-4-ylethoxy)phenyl]-4H-spiro[1,3-benzodioxine-2,4′-25 piperidine];
6-(2,4-dimethoxyphenyl)-4H-spiro[1,3-benzodioxine-2,4′-piperidine];
6-(3,4,5-trimethoxyphenyl)-4H-spiro[1,3-benzodioxine-2,4′-piperidine];
6-quinolin-5-yl-4H-spiro[1,3-benzodioxine-2,4′-piperidine];
N-[3-(4H-spiro[1,3-benzodioxine-2,4′-piperidin]-6-yl)phenyl]pyrazine-2-carboxamide;
2-(3,4-dimethoxyphenyl)-N-[3-(4H-spiro[1,3-benzodioxine-2,4′-piperidin]-6-yl)phenyl]acetamide;
N-[2-(3,4-dimethoxyphenyl)ethyl]-3-(4H-spiro[1,3-benzodioxine-2,4′-piperidin]-6-yl)benzamide;
3-(4H-spiro[1,3-benzodioxine-2,4′-piperidin]-6-yl)-N-(3,4,5-trimethoxybenzyl)benzamide
2-(2,3-dimethoxyphenyl)-N-[3-(4H-spiro[1,3-benzodioxine-2,4′-piperidin]-6-yl)phenyl]acetamide;
2-(2,4-dimethoxyphenyl)-N-[3-(4H-spiro[1,3-benzodioxine-2,4′-piperidin]-6-yl)phenyl]acetamide;
3-(1H-indol-3-yl)-N-[3-(4H-spiro[1,3-benzodioxine-2,4′-piperidin]-6-yl)phenyl]propanamide;
2-(5-methoxy-2-methyl-1H-indol-3-yl)-N-[3-(4H-spiro[1,3-benzodioxine-2,4′-piperidin]-6-yl)phenyl]acetamide;
2-(2-methyl-1H-indol-3-yl)-N-[3-(4H-spiro[1,3-benzodioxine-2,4′-piperidin]-6-yl)phenyl]acetamide;
2-(7-methyl-1H-indol-3-yl)-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-3-(3,4,5-trimethoxyphenyl)propanamide;
3-phenoxy-N-[3-(4H-spiro[1,3-benzodioxine-2,4′-piperidin]-6-yl)phenyl]propanamide
2-(3,5-dimethoxyphenyl)-N-[3-(4H-spiro[1,3-benzodioxine-2,4′-piperidin]-6-yl)phenyl]acetamide;
2-[4-(dimethylamino)phenyl]-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-2-(2,3,4-trimethoxyphenyl)acetamide;
2-(4-hydroxy-3,5-dimethoxyphenyl)-N-[3-(4H-spiro[1,3-benzodioxine-2,4′-piperidin]-6-yl)phenyl]acetamide;
2-(4-hydroxy-3-methoxyphenyl)-N-[3-(4H-spiro[1,3-benzodioxine-2,4′-piperidin]-6-yl)phenyl]acetamide
N-[3-(4H-spiro[1,3-benzodioxine-2,4′-piperidin]-6-yl)phenyl]-2-[3-(trifluoromethyl)phenyl]acetamide;
2-(6-methoxy-1-benzofuran-3-yl)-N-[3-(4H-spiro[1,3-benzodioxine-2,4′-piperidin]-6-yl)phenyl]acetamide;
N-[3-(4H-spiro[1,3-benzodioxine-2,4′-piperidin]-6-yl)phenyl]-N′-(3,4,5-trimethoxyphenyl)urea;
N-[3-(4H-spiro[1,3-benzodioxine-2,4′-piperidin]-6-yl)phenyl]-N′-(3,4,5-trimethoxybenzyl)urea;
N-(2-methylquinolin-6-yl)-N′-[3-(4H-spiro[1,3-benzodioxine-2,4′-piperidin]-6-yl)phenyl]urea;
N-[2-(5-methoxy-1H-indol-3-yl)ethyl]-3-(4H-spiro[1,3-benzodioxine-2,4′-piperidin]-6-yl)benzamide;
2-(5-methoxy-1H-indol-3-yl)-N-[2-methoxy-5-(4H-spiro[1,3-benzodioxine-2,4′-piperidin]-6-yl)phenyl]acetamide;
N-[2-methoxy-5-(4H-spiro[1,3-benzodioxine-2,4′-piperidin]-6-yl)phenyl]-2-(3,4,5-trimethoxyphenyl)acetamide;
3-(3,4-dimethoxyphenyl)-N-[2-methoxy-5-(4H-spiro[1,3-benzodioxine-2,4′-piperidin]-6-yl)phenyl]propanamide;
2-(5-hydroxy-1H-indol-3-yl)-N-[3-(4H-spiro[1,3-benzodioxine-2,4′-piperidin]-6-yl)phenyl]acetamide;
N-[2-(1H-indol-3-yl)ethyl]-3-(4H-spiro[1,3-benzodioxine-2,4′-piperidin]-6-yl)benzamide
N-[2-(6-methoxy-1H-indol-3-yl)ethyl]-3-(4H-spiro[1,3-benzodioxine-2,4′-piperidin]-6-yl)benzamide;
N-[2-(5-chloro-1H-indol-3-yl)ethyl]-3-(4H-spiro[1,3-benzodioxine-2,4′-piperidin]-6-yl)benzamide;
N-[2-(5-fluoro-1H-indol-3-yl)ethyl]-3-(4H-spiro[1,3-benzodioxine-2,4′-piperidin]-6-yl)benzamide;
N-[2-(5-methoxy-1H-indol-3-yl)ethyl]-5-(4H-spiro[1,3-benzodioxine-2,4′-piperidin]-6-yl)nicotinamide;
N-[5-(4H-spiro[1,3-benzodioxine-2,4′-piperidin]-6-yl)pyridin-3-yl]-2-(3,4,5-trimethoxyphenyl)acetamide;
2-(5-methoxy-1H-indol-3-yl)-N-[5-(4H-spiro[1,3-benzodioxine-2,4′-piperidin]-6-yl)pyridin-3-yl]acetamide;
N-[2-(5-methoxy-1H-indol-3-yl)ethyl]-4-(4H-spiro[1,3-benzodioxine-2,4′-piperidin]-6-yl)pyridine-2-carboxamide;
2-fluoro-5-(4H-spiro[1,3-benzodioxine-2,4′-piperidin]-6-yl)-N-(3,4,5-trimethoxybenzyl)benzamide;
2-fluoro-N-[2-(5-methoxy-1H-indol-3-yl)ethyl]-5-(4H-spiro[1,3-benzodioxine-2,4′-piperidin]-6-yl)benzamide;
2-(1H-benzimidazol-1-yl)-N-[3-(4H-spiro[1,3-benzodioxine-2,4′-piperidin]-6-yl)phenyl]acetamide;
2-(5-methyl-2-phenyl-1,3-thiazol-4-yl)-N-[3-(4H-spiro[1,3-benzodioxine-2,4′-piperidin]-6-yl)phenyl]acetamide;
2-(4-tert-butylphenyl)-N-[3-(4H-spiro[1,3-benzodioxine-2,4′-piperidin]-6-yl)phenyl]acetamide;
2-(5-ethyl-1H-indol-3-yl)-N-[3-(4H-spiro[1,3-benzodioxine-2,4′-piperidin]-6-yl)phenyl]acetamide;
3-(1H-benzimidazol-1-yl)-N-[3-(4H-spiro[1,3-benzodioxine-2,4′-piperidin]-6-yl)phenyl]propanamide;
2-(2H-1,2,3-benzotriazol-2-yl)-N-[3-(4H-spiro[1,3-benzodioxine-2,4′-piperidin]-6-yl)phenyl]acetamide;
3-(2-phenyl-1H-imidazol-1-yl)-N-[3-(4H-spiro[1,3-benzodioxine-2,4′-piperidin]-6-yl)phenyl]propanamide;
3-(2,3-dihydro-1-benzofuran-5-yl)-N-[3-(4H-spiro[1,3-benzodioxine-2,4′-piperidin]-6-yl)phenyl]propanamide;
2-(3-pyridin-3-ylphenyl)-N-[3-(4H-spiro[1,3-benzodioxine-2,4′-piperidin]-6-yl)phenyl]acetamide;
N-[3-(1′-isopropyl-4H-spiro[1,3-benzodioxine-2,4′-piperidin]-6-yl)phenyl]-2-(3,4,5-trimethoxyphenyl)acetamide;
3-(3-chloro-4-methoxyphenyl)-N-[3-(4H-spiro[1,3-benzodioxine-2,4′-piperidin]-6-yl)phenyl]propanamide;
2-(1H-benzimidazol-2-yl)-N-[3-(4H-spiro[1,3-benzodioxine-2,4′-piperidin]-6-yl)phenyl]acetamide;
3-(1H-1,2,3-benzotriazol-1-yl)-N-[3-(4H-spiro[1,3-benzodioxine-2,4′-piperidin]-6-yl)phenyl]propanamide;
3-(1H-indazol-1-yl)-N-[3-(4H-spiro[1,3-benzodioxine-2,4′-piperidin]-6-yl)phenyl]propanamide;
2-(2-methyl-1H-benzimidazol-1-yl)-N-[3-(4H-spiro[1,3-benzodioxine-2,4′-piperidin]-6-yl)phenyl]acetamide;
3-(2-methyl-1H-benzimidazol-1-yl)-N-[3-(4H-spiro[1,3-benzodioxine-2,4′-piperidin]-6-yl)phenyl]propanamide;
2-pyridin-2-yl-N-[3-(4H-spiro[1,3-benzodioxine-2,4′-piperidin]-6-yl)phenyl]acetamide;
2-pyridin-4-yl-N-[3-(4H-spiro[1,3-benzodioxine-2,4′-piperidin]-6-yl)phenyl]acetamide;
3-(1H-benzimidazol-2-yl)-N-[3-(4H-spiro[1,3-benzodioxine-2,4′-piperidin]-6-yl)phenyl]propanamide;
N-methyl-N-(2-oxo-2-{[3-(4H-spiro[1,3-benzodioxine-2,4′-piperidin]-6-yl)phenyl]amino}ethyl)benzamide;
N-[2-(3,4-dimethoxyphenyl)ethyl]-2-methyl-3-(4H-spiro[1,3-benzodioxine-2,4′-piperidin]-6-yl)benzamide; and
2-methyl-3-(4H-spiro[1,3-benzodioxine-2,4′-piperidin]-6-yl)-N-(3,4,5-trimethoxybenzyl)benzamide.

17. A method for treating an orexin-1 receptor-related disorder or an orexin-2 receptor-related disorder, the method comprising administering the compound of claim 1.

18. The method of claim 17, wherein the disorder is selected from obesity and related disorders such as diabetes type II, dyslipidemia and the metabolic syndrome, cardiovascular diseases such as atherosclerotic vascular disease, angina pectoris, myocardial infarction and stroke, and sleeping disorders.

19. A pharmaceutical formulation comprising a compound according to claim 1 as active ingredient, in combination with a pharmaceutically acceptable diluent or carrier.
